(19) United States
(12) Patent Application Publication
(10) Pub. No.: US 2024/0293323 A1
(43) Pub. Date: Sep. 5, 2024

(54) PHARMACEUTICAL COMPOSITIONS OF AN EPIDERMAL GROWTH FACTOR RECEPTOR INHIBITOR

(71) Applicant: Blueprint Mdeicines Coroporation, Cambridge, MA (US)

(72) Inventors: Caitlin N. Kinkema, Cambridge, MA (US); Debra L. Mazaik, Cambridge, MA (US); Clare Aubrey Medendorp, Cambridge, MA (US); Onkar Shripad Vaze, Cambridge, MA (US)

(21) Appl. No.: 18/573,229

(22) PCT Filed: Jun. 22, 2022
(86) PCT No.: PCT/US2022/034433

§ 371 Date: Dec. 21, 2023

Related U.S. Application Data
(60) Provisional application No. 63/214,099, filed on Jun. 23, 2021.

(51) Int. Cl. A61K 9/20; A61K 9/28; A61K 9/48; A61K 31/506

(52) U.S. Cl. CPC A61K 9/2009; A61K 9/2018; A61K 9/2031; A61K 9/2054; A61K 9/284; A61K 9/485; A61K 9/4858; A61K 9/4866; A61K 31/506

(57) ABSTRACT

The present disclosure relates to pharmaceutical compositions comprising an intragranular phase, wherein the intragranular phase comprises: (i) an amorphous solid dispersion comprising Compound (I) or a pharmaceutically acceptable salt thereof, and a pharmaceutically acceptable polymer, and (ii) a surfactant; and an extragranular phase, wherein the extragranular phase comprises a surfactant. The present disclosure also relates to methods of using said compositions in the treatment of various disorders.

### CROSS-REFERENCE TO RELATED APPLICATIONS

This application claims priority to U.S. Provisional Application No. 63/214,099, filed Jun. 23, 2021. The entire contents of the aforementioned application are incorporated herein by reference.

### BACKGROUND

Compound (I), the structure of which is shown below, is disclosed as one of many EGFR inhibitors compounds in patent application no. PCT/US2020/066629. Compound (I) is a potent and selective EGFR inhibitor provided in an oral dosage form to selectively target oncogenic EGFR mutations in certain cancer patients, including patients having a cancer harboring EGFR with one or more alterations, including L858R and/or exon 19 deletion mutation, T790M mutation, and/or C797S mutation. Compound (I) can also be referred to as: N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine, and has the following chemical structure:

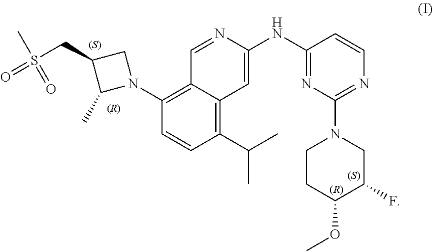

There is a need to develop pharmaceutical compositions of Compound (I) that are suitable for medical use.

### SUMMARY

The present disclosure features pharmaceutical compositions comprising an intragranular phase, wherein the intragranular phase comprises: (i) an amorphous solid dispersion comprising Compound (I) or a pharmaceutically acceptable salt thereof, and a pharmaceutically acceptable polymer, and (ii) a surfactant; and an extragranular phase, wherein the extragranular phase comprises at least one of the following: a surfactant, a disintegrant, a glidant, a lubricant, and a filler.

### BRIEF DESCRIPTIONS OF DRAWINGS

FIG. 1 is a comparison of the dissolution profile for Formulation 1 and Formulation 2 as compared to the 50:50 Compound (I):HPMCAS-M alone (i.e., the ASD alone) in biorelevant media i.e. at gastric pH and intestinal pH.

FIG. 2 is a comparison of the dissolution profile for Formulation 3 as compared to the 50:50 Compound (I):HPMCAS-M alone (i.e., the ASD alone) in biorelevant media i.e. at gastric pH and intestinal pH.

FIG. 3 is a comparison of the dissolution profile for Formulation 4 and Formulation 5 as compared to the 50:50 Compound (I):HPMCAS-M alone (i.e., the ASD alone) in biorelevant media i.e. at gastric pH and intestinal pH.

FIG. 4 is a comparison of dog PK plasma concentration profiles between 50:50 Compound (I):HPMCAS-M alone (i.e., the ASD alone), Tablet formulation T1 and T2 and T3.

### DETAILED DESCRIPTIONS

It was discovered that Compound (I) is a potent kinase inhibitor but has a low aqueous solubility. To enhance the bioavailability, Compound (I) was molecularly dispersed into a polymer matrix system, creating a solid dispersion, which improves the rate of dissolution. While dissolving a poorly water soluble active pharmaceutical ingredient (API) in a polymer improves the overall solubility, depending on the polymer matrix, it can delay the onset of API dissolution since the API needs to be released from the matrix. The properties of the excipients further impact the dissolution behavior of the solid dispersion. Yet, it was unexpected to find that the addition of a surfactant in an intragranular phase, and an extragranular phase comprised of at least one of a surfactant, a disintegrant, a glidant, a lubricant, and a filler of a pharmaceutical composition allows for wetting, dispersion, and dissolution of Compound (I) at a rate suitable for an immediate release solid dosage form.

### Pharmaceutical Compositions

“Extragranular” is of or pertaining to additional excipients that are incorporated in a formulation after granulation, i.e., ingredients that are located externally to a granule structure.

“Intragranular” is of or pertaining to additional excipients that are incorporated in a formulation prior to granulation, i.e., ingredients that are located internally in a granule structure.

A “pharmaceutically acceptable polymer” may be a nonionic polymer or an ionic polymer. In general, when selecting, polymers should be selected based on their polymer chemistry with the properties of the API and manufacturing aspects of the formulation. Polymers suitable for use in an amorphous solid dispersion of the present disclosure include, but are not limited to, homopolymers or copolymers of N-vinyl lactams, such as homopolymers or copolymers of N-vinyl pyrrolidone (e.g., polyvinylpyrrolidone (PVP), or copolymers of N-vinyl pyrrolidone and vinyl acetate (PVPVA) or vinyl propionate); cellulose esters or cellulose ethers, such as alkylcelluloses (e.g., methylcellulose or ethylcellulose), hydroxyalkylcelluloses (e.g., hydroxypropylcellulose), hydroxyalkylalkylcelluloses (e.g., hydroxypropylmethylcellulose), and cellulose phthalates or succinates (e.g., cellulose acetate phthalate and hydroxypropylmethylcellulose phthalate, hydroxypropylmethylcellulose succinate, or hydroxypropylmethylcellulose acetate succinate); high molecular polyalkylene oxides, such as polyethylene oxide, polypropylene oxide, and copolymers of ethylene oxide and propylene oxide; polyacrylates or polymethacrylates, such as methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), and poly(hydroxyalkyl methacrylates); polyacrylamides; vinyl acetate polymers, such as copolymers of vinyl acetate and crotonic acid, and partially hydrolyzed polyvinyl acetate (also referred to as partially saponified “polyvinyl alcohol”); polyvinyl alcohol; oligo- or polysaccharides, such as carrageenans, galactomannans, and xanthan gum; polyhydroxyalkylacrylates; polyhydroxyalkyl-methacrylates; copolymers of methyl methacrylate and acrylic acid; polyethylene glycols (PEGs), including polyvinyl graph coploymer; or any mixture thereof. In one aspect, the polymer is hydroxypropyl methylcellulose (HPMC or hypromellose), hydroxypropyl methylcellulose acetate succinate (HPMC-AS), including HPMCAS-M, HPMCAS-L, and HPMCAS-H) hydroxypropylmethylcellulose E5 (HPMC-E5), hydroxypropylmethylcellulose E3 (HPMC-E3), HPMCAS-M, HPMC E3LV, HPMCP-HP55, vinylpyrrolidone-vinyl acetate copolymer (KOLLIDON VA64 or KOLLIDON K30), dimethylaminoethyl methacrylate-copolymer (EUDRAGIT EPO), Eudragrit 100, Eudragrit L, poly(ethylene) oxide (POLYOX), or polyvinyl caprolactam-polyvinyl acetate-polyethylene glycol graft copolymer (SOLUPLUS). In a particular aspect, the polymer is 6:4 linear random copolymer of N-vinylpyrrolidone and vinyl acetate (e.g., PVPVA-64), HPMCAS-M, Eudragit L100-55, Eudragit L100, and HPMC E3LV. In a particular aspect, the polymer is PVPVA-64, and HPMCAS. In a particular aspect, the polymer is HPMCAS. In a particular aspect, the polymer is PVPVA-64, and HPMCAS-M. In a particular aspect, the polymer is HPMCAS-M. In a particular aspect, the polymer is HPMCAS-MG.

HPMC E3 refers to hydroxypropylmethylcellulose having a viscosity of about 2.4-3.6 mPa s, (2% in water)). HPMC E5 refers to hydroxypropylmethylcellulose having a viscosity of about 4 to 6 mPa s (2% in water). HPMCAS-M, HPMCAS-L, and HPMCAS-H refers to hydroxypropyl methyl cellulose acetate succinate, in which the M, L, and H are different grades of polymer and vary in acetyl and succinoyl content. HPMC E3LV refers to low viscosity hydroxypropyl methyl cellulose and E3 is the grade which is determined by the average content of methoxyl groups and hydroxypropyl groups. In a particular aspect, the polymer is hydroxypropyl methylcellulose acetate succinate. In a particular aspect, the hydrophilic polymer is hydroxypropyl methylcellulose acetate succinate (HPMC-AS).

In one aspect, the present disclosure features an amorphous solid dispersion comprising Compound (I) or a pharmaceutically acceptable salt thereof, and a pharmaceutically acceptable polymer. In some embodiments, the polymer is hydroxypropyl methylcellulose (HPMC) (e.g., HPMCAS-M) or polyvinylpyrrolidone (PVP) or 6:4 linear random copolymer of N-vinylpyrrolidone and vinyl acetate (e.g., PVPVA-64).

In one aspect, the solvent used to prepare the amorphous solid dispersion is dichloromethane (DCM), methanol (MeOH), or a combination thereof. In some embodiments, the ratio of dichloromethane to methanol is 100:0 (e.g., DCM only); 90:10 DCM:MeOH; 80:20 DCM:MeOH; 70:30 DCM:MeOH; or 60:40 DCM:MeOH.

In one aspect, the solvent used to prepare the amorphous solid dispersion is acetone or water, or a combination thereof. In some embodiments, the ratio of acetone to water is 100:0 acetone:water (e.g., 100% acetone), 95:5 acetone:water, or 90:10 acetone:water.

In one aspect, the solids content (i.e., the total concentration w/w of Compound (I) and the polymer in the solvent used to prepare the amorphous solid dispersion) of the spray solution to prepare the amorphous solid dispersion is at 4% w/w to 15% w/w, for example, 4% w/w, 4.5% w/w, 5% w/w, 5.5% w/w, 6% w/w, 6.5% w/w, 7.0% w/w, 7.5% w/w, 8.0% w/w, 8.5% w/w, 9% w/w, 9.5% w/w, 10% w/w, 10.5% w/w, 11% w/w, 11.5% w/w, 12% w/w, 12.5% w/w, 13% w/w, 13.5% w/w, 14% w/w, 14.5% w/w, or 15% w/w.

In one aspect, the Compound (I) free base, or an equivalent amount of a pharmaceutically acceptable salt thereof, and the polymer are in a weight percent ratio of about 1:1, for example, a disclosed composition may include about 100 mg Compound (I) free base and about 100 mg polymer (or 50 mg Compound (I) and about 50 mg polymer or 25 mg Compound (I) and about 25 mg polymer) such as disclosed herein. In another aspect, the Compound (I) free base, or an equivalent amount of a pharmaceutically acceptable salt thereof, and the polymer are in a weight percent ratio of from about 1:5 to about 5:1. In another aspect, the Compound (I) free base, or an equivalent amount of a pharmaceutically acceptable salt thereof, and the polymer are in a weight percent ratio of from about 1:3 to about 3:1. In another aspect, the Compound (I) free base, or an equivalent amount of a pharmaceutically acceptable salt thereof, and the polymer are in a weight percent ratio of from about 1:2 to about 2:1. In another aspect, the Compound (I) free base, or an equivalent amount of a pharmaceutically acceptable salt thereof, and the polymer are in a weight percent ratio of from about 1:1.5 to about 1.5:1. In another aspect, the Compound (I) free base, or an equivalent amount of a pharmaceutically acceptable salt thereof, and the polymer are in a weight percent ratio of from about 1:1.1 to about 1.1:1. In another aspect, the Compound (I) free base, or an equivalent amount of a pharmaceutically acceptable salt thereof, and the polymer are in a weight percent ratio of about 2:3. In another aspect, the Compound (I) free base, or an equivalent amount of a pharmaceutically acceptable salt thereof, and the polymer are in a weight percent ratio of about 3:7.

In one aspect, the Compound (I) free base, or an equivalent amount of a pharmaceutically acceptable salt thereof, and the polymer are in a weight percent ratio of about 10%:90%, 20%:80%, about 30%:70%, 40%:60%, 50%:50%, about 60%:40%, about 70%:30%, about 80%:20%, or about 90%:10%. In a particular aspect, the polymer is PVP-VA, Eudragit 100, HPMCAS-M, HPMCAS-MG, or HPMC E3LV and is at about 70%, about 60%, and about 50% and the Compound (I) free base is at about 30%, about 40, and about 50% drug loading.

In one aspect, a composition disclosed herein is prepared in an oral dosage form.

In one aspect, a composition or an oral dosage form as described herein comprises the amorphous solid dispersion from about 20% to about 80% by weight of the composition or the oral dosage form, based on the total weight of the composition or the oral dosage form. In one aspect, a composition or an oral dosage form as described herein comprises the amorphous solid dispersion from about 25% to about 75% by weight of the composition or the oral dosage form, based on the total weight of the composition or the oral dosage form. In one aspect, a composition or an oral dosage form as described herein comprises the amorphous solid dispersion from about 35% to about 65% by weight of the composition or the oral dosage form, based on the total weight of the composition or the oral dosage form. In one aspect, a composition or an oral dosage form as described herein comprises the amorphous solid dispersion from about 45% to about 55% by weight of the composition or the oral dosage form, based on the total weight of the composition or the oral dosage form. In one aspect, a composition or an oral dosage form as described herein comprises the amorphous solid dispersion from about 40% to about 50% by weight of the composition or the oral dosage form, based on the total weight of the composition or the oral dosage form. In one aspect, a composition or an oral dosage form as described herein comprises the amorphous solid dispersion at about 25%, about 30%, about 35%, about 40%, about 45%, about 50%, about 55%, about 60%, about 65%, about 70%, or about 75% by weight of the composition or the oral dosage form, based on the total weight of the composition or the oral dosage form. In one aspect, the amorphous solid dispersion is in the intragranular phase.

In one aspect, the amorphous solid dispersion is prepared by hot melt extrusion, lyophilization, co-precipitation, spray drying, hot melt congealing, solvent casting, or melt quenching. The solid dispersions prepared from these different methods may therefore differ in properties based on the matrix formed with the dispersion polymer, such as porosity, surface area, density, stability, hygroscopicity, dissolution and therefore bioavailability. In a particular aspect, the amorphous solid dispersion is prepared by spray drying.

In one aspect, the composition or solid dosage form comprises a surfactant. Non-limiting examples of suitable surfactants include cationic, anionic, zwitterionic, or non-ionic and mixtures thereof. Non-limiting examples of surfactants that can be used include quaternary ammonium compounds, for example dioctyl sodium sulfosuccinate, polyoxyethylene alklphenyl ethers, for example nonoxynol 9, nonoxynol 10 and octoxynol 9, Poloxamers (poloxyethylene and polypropylene block copolymers, such as Poloxamer 407), polyoxyethylene fatty acid glycerides and oils, for example polyoxyethylene (8), caprylic/capric mono- and diglycerides, polyoxyethylene (35) castor oil and polyoxyethylene (40) hydrogenated castor oil, polyethylene alkyl ethers, for example poloxyethylene (20) cetostearyl ether, polyoxyethelene fatty acid esters, for example polyoxyethylene (40) stearate, polyoxyethylene sorbitan esters, for example polysorbate 20 and polysorbate 80 (e.g. Tween 80), propylene glycol fatty esters, for example propylene glycol laurate, sodium lauryl sulfate, fatty acids and salts thereof, for example oleic acid, sodium oleate and triethanolamine oleate, glyceryl fatty acid esters, for example sorbitan monolaurate, sorbitan monooleate, sorbitan monopalmitate and sorbitan monostearate, tyloxapol, and mixtures thereof. In a particular aspect, the surfactant is a poloxamer such as poloxamer 407. In a more particular aspect, the surfactant is poloxamer 407 micro with an average particle size of approximately 50 μm.

In one aspect, a composition or an oral dosage form as described herein comprises the surfactant from about 0.25% to about 20% by weight of the composition or the oral dosage form, based on the total weight of the composition or the oral dosage form. In one aspect, a composition or an oral dosage form as described herein comprises the surfactant from about 0.25% to about 15% by weight of the composition or the oral dosage form, based on the total weight of the composition or the oral dosage form. In one aspect, a composition or an oral dosage form as described herein comprises the surfactant from about 0.25% to about 10% by weight of the composition or the oral dosage form, based on the total weight of the composition or the oral dosage form. In one aspect, a composition or an oral dosage form as described herein comprises the surfactant about 0.25% to about 5% by weight of the composition or the oral dosage form, based on the total weight of the composition or the oral dosage form. In one aspect, a composition or an oral dosage form as described herein comprises the surfactant about 0.25% to about 3% by weight of the composition or the oral dosage form, based on the total weight of the composition or the oral dosage form. In one aspect, a composition or an oral dosage form as described herein comprises the surfactant in about 0.25%, about 0.4%, about 0.5%, about 0.75%, about 1%, about 1.25%, about 1.5%, about 1.75%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight of the composition or the oral dosage form, based on the total weight of the composition or the oral dosage form.

In one aspect, a composition or an oral dosage form as described herein comprises the surfactant in an intragranular phase from about 0.1% to about 20% by weight of the composition or the oral dosage form, based on the total weight of the composition or the oral dosage form, and in an extragranular phase from about 0.1% to about 20% by weight of the composition or the oral dosage form, based on the total weight of the composition or the oral dosage form, wherein the total amount of surfactant in the combined intragranular and extragranular phases in the composition or an oral dosage is from about 0.25% to about 20% by weight of the composition or the oral dosage form, based on the total weight of the composition or the oral dosage form.

In one aspect, a composition or an oral dosage form as described herein comprises the surfactant in an intragranular phase from about 0.1% to about 15% by weight of the composition or the oral dosage form, based on the total weight of the composition or the oral dosage form, and in an extragranular phase from about 0.1% to about 15% by weight of the composition or the oral dosage form, based on the total weight of the composition or the oral dosage form, wherein the total amount of surfactant in the combined intragranular and extragranular phases in the composition or an oral dosage is from about 0.25% to about 15% by weight of the composition or the oral dosage form, based on the total weight of the composition or the oral dosage form.

In one aspect, a composition or an oral dosage form as described herein comprises the surfactant in an intragranular phase from about 0.1% to about 10% by weight of the composition or the oral dosage form, based on the total weight of the composition or the oral dosage form, and in an extragranular phase from about 0.1% to about 10% by weight of the composition or the oral dosage form, based on the total weight of the composition or the oral dosage form, wherein the total amount of surfactant in the combined intragranular and extragranular phases in the composition or an oral dosage is from about 0.25% to about 10% by weight of the composition or the oral dosage form, based on the total weight of the composition or the oral dosage form.

In one aspect, a composition or an oral dosage form as described herein comprises the surfactant in an intragranular phase from about 0.1% to about 5% by weight of the composition or the oral dosage form, based on the total weight of the composition or the oral dosage form, and in an extragranular phase from about 0.1% to about 5% by weight of the composition or the oral dosage form, based on the total weight of the composition or the oral dosage form, wherein the total amount of surfactant in the combined intragranular and extragranular phases in the composition or an oral dosage is from about 0.25% to about 5% by weight of the composition or the oral dosage form, based on the total weight of the composition or the oral dosage form.

In one aspect, a composition or an oral dosage form as described herein comprises the surfactant in an intragranular phase from about 0.1% to about 3% by weight of the composition or the oral dosage form, based on the total weight of the composition or the oral dosage form, and in an extragranular phase from about 0.1% to about 2% by weight of the composition or the oral dosage form, based on the total weight of the composition or the oral dosage form, wherein the total amount of surfactant in the combined intragranular and extragranular phases in the composition or an oral dosage is from about 0.25% to about 5% by weight of the composition or the oral dosage form, based on the total weight of the composition or the oral dosage form.

In one aspect, a composition or an oral dosage form as described herein comprises the surfactant in the intragranular phase from about 0.1% to about 20% by weight of the composition or the oral dosage form, based on the total weight of the composition or the oral dosage form. In one aspect, a composition or an oral dosage form as described herein comprises the surfactant in the intragranular phase from about 0.1% to about 15% by weight of the composition or the oral dosage form, based on the total weight of the composition or the oral dosage form. In one aspect, a composition or an oral dosage form as described herein comprises the surfactant in the intragranular phase from about 0.1% to about 10% by weight of the composition or the oral dosage form, based on the total weight of the composition or the oral dosage form. In one aspect, a composition or an oral dosage form as described herein comprises the surfactant in the intragranular phase from about 0.1% to about 5% by weight of the composition or the oral dosage form, based on the total weight of the composition or the oral dosage form. In one aspect, a composition or an oral dosage form as described herein comprises the surfactant in the intragranular phase from about 0.1% to about 3% by weight of the composition or the oral dosage form, based on the total weight of the composition or the oral dosage form. In one aspect, a composition or an oral dosage form as described herein comprises the surfactant in the intragranular phase from about 0.1% to about 2% by weight of the composition or the oral dosage form, based on the total weight of the composition or the oral dosage form. In one aspect, a composition or an oral dosage form as described herein comprises the surfactant in the intragranular phase at about 0.1%, about 0.25%, about 0.5%, about 0.75%, about 1%, about 1.25%, about 1.5%, about 1.75%, about 2%, about 2.25%, about 2.5%, about 2.75%, or about 3% by weight of the composition or the oral dosage form, based on the total weight of the composition or the oral dosage form.

In one aspect, a composition or an oral dosage form as described herein comprises the surfactant in the extragranular phase from about 0% to about 20% by weight of the composition or the oral dosage form, based on the total weight of the composition or the oral dosage form. In one aspect, a composition or an oral dosage form as described herein comprises the surfactant in the extragranular phase from about 0% to about 15% by weight of the composition or the oral dosage form, based on the total weight of the composition or the oral dosage form. In one aspect, a composition or an oral dosage form as described herein comprises the surfactant in the extragranular phase from about 0% to about 10% by weight of the composition or the oral dosage form, based on the total weight of the composition or the oral dosage form. In one aspect, a composition or an oral dosage form as described herein comprises the surfactant in the extragranular phase from about 0% to about 5% by weight of the composition or the oral dosage form, based on the total weight of the composition or the oral dosage form. In one aspect, a composition or an oral dosage form as described herein comprises the surfactant in the extragranular phase from about 0.1% to about 2% by weight of the composition or the oral dosage form, based on the total weight of the composition or the oral dosage form. In one aspect, a composition or an oral dosage form as described herein comprises the surfactant in the extragranular phase at 0%, about 0.1%, about 0.25%, about 0.5%, about 0.75%, about 1%, about 1.25%, about 1.5%, about 1.75%, about 2%, about 2.25%, about 2.5%, about 2.75%, or about 3% by weight of the composition or the oral dosage form, based on the total weight of the composition or the oral dosage form.

In one aspect, the composition or solid dosage form further comprises a disintegrant. Non-limiting examples of suitable disintegrant include, either individually or in combination, starches, including sodium starch glycolate and pregelatinized corn starches, clays, celluloses such as purified cellulose, microcrystalline cellulose, methylcellulose, carboxymethylcellulose and sodium carboxymethylcellulose, croscarmellose sodium, alginates, crospovidone, and gums such as agar, guar, locust bean, karaya, pectin and tragacanth gums. In a particular aspect, the disintegrant is crospovidone. In some embodiments, disintegrants may be added at any suitable step during the preparation of the composition, particularly prior to granulation or during a lubrication step prior to compression.

In one aspect, a composition or an oral dosage form as described herein comprises the disintegrant from about 0% to about 30% by weight of the composition or the oral dosage form, based on the total weight of the composition or the oral dosage form. In one aspect, a composition or an oral dosage form as described herein comprises the disintegrant from about 0% to about 20% by weight of the composition or the oral dosage form, based on the total weight of the composition or the oral dosage form. In one aspect, a composition or an oral dosage form as described herein comprises the disintegrant from about 0% to about 10% by weight of the composition or the oral dosage form, based on the total weight of the composition or the oral dosage form. In one aspect, a composition or an oral dosage form as described herein comprises the disintegrant from about 1% to about 10% by weight of the composition or the oral dosage form, based on the total weight of the composition or the oral dosage form. In one aspect, a composition or an oral dosage form as described herein comprises the disintegrant from about 1% to about 7% by weight of the composition or the oral dosage form, based on the total weight of the composition or the oral dosage form. In one aspect, a composition or an oral dosage form as described herein comprises the disintegrant from about 5% to about 10% by weight of the composition or the oral dosage form, based on the total weight of the composition or the oral dosage form. In one aspect, a composition or an oral dosage form as described herein comprises the disintegrant at about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight of the composition or the oral dosage form, based on the total weight of the composition or the oral dosage form.

In one aspect, a composition or an oral dosage form as described herein comprises the disintegrant in an intragranular phase from about 0% to about 30% by weight of the composition or the oral dosage form, based on the total weight of the composition or the oral dosage form, and in an extragranular phase from about 0% to about 30% by weight of the composition or the oral dosage form, based on the total weight of the composition or the oral dosage form, wherein the total amount of disintegrant in the combined intragranular and extragranular phases in the composition or an oral dosage is from about 0.2% to about 30% by weight of the composition or the oral dosage form, based on the total weight of the composition or the oral dosage form.

In one aspect, a composition or an oral dosage form as described herein comprises the disintegrant in an intragranular phase from about 0% to about 20% by weight of the composition or the oral dosage form, based on the total weight of the composition or the oral dosage form, and an in extragranular phase from about 0% to about 20% by weight of the composition or the oral dosage form, based on the total weight of the composition or the oral dosage form, wherein the total amount of disintegrant in the combined intragranular and extragranular phases in the composition or an oral dosage is from about 0.2% to about 20% by weight of the composition or the oral dosage form, based on the total weight of the composition or the oral dosage form.

In one aspect, a composition or an oral dosage form as described herein comprises the disintegrant in an intragranular phase from about 0% to about 10% by weight of the composition or the oral dosage form, based on the total weight of the composition or the oral dosage form, and in an extragranular phase from about 0% to about 10% by weight of the composition or the oral dosage form, based on the total weight of the composition or the oral dosage form, wherein the total amount of disintegrant in the combined intragranular and extragranular phases in the composition or an oral dosage is from about 0.2% to about 10% by weight of the composition or the oral dosage form, based on the total weight of the composition or the oral dosage form.

In one aspect, a composition or an oral dosage form as described herein comprises the disintegrant in an intragranular phase from about 0% to about 10% by weight of the composition or the oral dosage form, based on the total weight of the composition or the oral dosage form, and in an extragranular phase from about 0% to about 10% by weight of the composition or the oral dosage form, based on the total weight of the composition or the oral dosage form, wherein the total amount of disintegrant in the combined intragranular and extragranular phases in the composition or an oral dosage is from about 1% to about 10% by weight of the composition or the oral dosage form, based on the total weight of the composition or the oral dosage form.

In one aspect, a composition or an oral dosage form as described herein comprises the disintegrant in an intragranular phase from about 0% to about 7% by weight of the composition or the oral dosage form, based on the total weight of the composition or the oral dosage form, and in an extragranular phase from about 0% to about 7% by weight of the composition or the oral dosage form, based on the total weight of the composition or the oral dosage form, wherein the total amount of disintegrant in the combined intragranular and extragranular phases in the composition or an oral dosage is from about 1% to about 7% by weight of the composition or the oral dosage form, based on the total weight of the composition or the oral dosage form.

In one aspect, a composition or an oral dosage form as described herein comprises the disintegrant in an intragranular phase from about 0% to about 10% by weight of the composition or the oral dosage form, based on the total weight of the composition or the oral dosage form, and in an extragranular phase from about 0% to about 10% by weight of the composition or the oral dosage form, based on the total weight of the composition or the oral dosage form, wherein the total amount of disintegrant in the combined intragranular and extragranular phases in the composition or an oral dosage is from about 5% to about 10% by weight of the composition or the oral dosage form, based on the total weight of the composition or the oral dosage form.

In one aspect, a composition or an oral dosage form as described herein comprises the disintegrant in an intragranular phase from about 0.1% to about 5% by weight of the composition or the oral dosage form, based on the total weight of the composition or the oral dosage form, and in an extragranular phase from about 0.1% to about 5% by weight of the composition or the oral dosage form, based on the total weight of the composition or the oral dosage form, wherein the total amount of disintegrant in the combined intragranular and extragranular phases in the composition or an oral dosage is from about 5.1% to about 10% by weight of the composition or the oral dosage form, based on the total weight of the composition or the oral dosage form.

In one aspect, a composition or an oral dosage form as described herein comprises the disintegrant in an intragranular phase from about 2% to about 5% by weight of the composition or the oral dosage form, based on the total weight of the composition or the oral dosage form, and in an extragranular phase from about 2% to about 5% by weight of the composition or the oral dosage form, based on the total weight of the composition or the oral dosage form, wherein the total amount of disintegrant in the combined intragranular and extragranular phases in the composition or an oral dosage is from about 4% to about 10% by weight of the composition or the oral dosage form, based on the total weight of the composition or the oral dosage form.

In one aspect, a composition or an oral dosage form as described herein comprises the disintegrant in the intragranular phase from about 0% to about 30% by weight of the composition or the oral dosage form, based on the total weight of the composition or the oral dosage form. In one aspect, a composition or an oral dosage form as described herein comprises the disintegrant in the intragranular phase from about 0% to about 20% by weight of the composition or the oral dosage form, based on the total weight of the composition or the oral dosage form. In one aspect, a composition or an oral dosage form as described herein comprises the disintegrant in the intragranular phase from about 0% to about 10% by weight of the composition or the oral dosage form, based on the total weight of the composition or the oral dosage form. In one aspect, a composition or an oral dosage form as described herein comprises the disintegrant in the intragranular phase from about 0.2% to about 10% by weight of the composition or the oral dosage form, based on the total weight of the composition or the oral dosage form. In one aspect, a composition or an oral dosage form as described herein comprises the disintegrant in the intragranular phase from about 1% to about 10% by weight of the composition or the oral dosage form, based on the total weight of the composition or the oral dosage form. In one aspect, a composition or an oral dosage form as described herein comprises the disintegrant in the intragranular phase from about 0% to about 5% by weight of the composition or the oral dosage form, based on the total weight of the composition or the oral dosage form. In one aspect, a composition or an oral dosage form as described herein comprises the disintegrant in the intragranular phase from about 0.1% to about 5% by weight of the composition or the oral dosage form, based on the total weight of the composition or the oral dosage form. In one aspect, a composition or an oral dosage form as described herein comprises the disintegrant in the intragranular phase from about 1% to about 5% by weight of the composition or the oral dosage form, based on the total weight of the composition or the oral dosage form. In one aspect, a composition or an oral dosage form as described herein comprises the disintegrant in the intragranular phase at about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight of the composition or the oral dosage form, based on the total weight of the composition or the oral dosage form.

In one aspect, a composition or an oral dosage form as described herein comprises the disintegrant in the extragranular phase from about 0% to about 30% by weight of the composition or the oral dosage form, based on the total weight of the composition or the oral dosage form. In one aspect, a composition or an oral dosage form as described herein comprises the disintegrant in the extragranular phase from about 0% to about 20% by weight of the composition or the oral dosage form, based on the total weight of the composition or the oral dosage form. In one aspect, a composition or an oral dosage form as described herein comprises the disintegrant in the extragranular phase from about 0% to about 10% by weight of the composition or the oral dosage form, based on the total weight of the composition or the oral dosage form. In one aspect, a composition or an oral dosage form as described herein comprises the disintegrant in the extragranular phase from about 0.2% to about 10% by weight of the composition or the oral dosage form, based on the total weight of the composition or the oral dosage form. In one aspect, a composition or an oral dosage form as described herein comprises the disintegrant in the extragranular phase from about 1% to about 10% by weight of the composition or the oral dosage form, based on the total weight of the composition or the oral dosage form. In one aspect, a composition or an oral dosage form as described herein comprises the disintegrant in the extragranular phase from about 0% to about 5% by weight of the composition or the oral dosage form, based on the total weight of the composition or the oral dosage form. In one aspect, a composition or an oral dosage form as described herein comprises the disintegrant in the extragranular phase from about 0.1% to about 5% by weight of the composition or the oral dosage form, based on the total weight of the composition or the oral dosage form. In one aspect, a composition or an oral dosage form as described herein comprises the disintegrant in the extragranular phase from about 1% to about 5% by weight of the composition or the oral dosage form, based on the total weight of the composition or the oral dosage form. In one aspect, a composition or an oral dosage form as described herein comprises the disintegrant in the extragranular phase at about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight of the composition or the oral dosage form, based on the total weight of the composition or the oral dosage form.

In one aspect, the composition or solid dosage form further comprises a glidant. Glidants can be used to promote powder flow of a solid formulation. Suitable glidants include colloidal silicon dioxide, starch, talc, tribasic calcium phosphate, powdered cellulose and magnesium trisilicate, and mixtures thereof. In a particular aspect, the glidant is colloidal silicon dioxide.

In one aspect, a composition or an oral dosage form as described herein comprises the glidant from about 0% to about 5% by weight of the composition or the oral dosage form, based on the total weight of the composition or the oral dosage form. In one aspect, a composition or an oral dosage form as described herein comprises the glidant from about 0% to about 4% by weight of the composition or the oral dosage form, based on the total weight of the composition or the oral dosage form. In one aspect, a composition or an oral dosage form as described herein comprises the glidant from about 0% to about 3% by weight of the composition or the oral dosage form, based on the total weight of the composition or the oral dosage form. In one aspect, a composition or an oral dosage form as described herein comprises the glidant from about 0% to about 2% by weight of the composition or the oral dosage form, based on the total weight of the composition or the oral dosage form. In one aspect, a composition or an oral dosage form as described herein comprises the glidant from about 0.5% to about 2% by weight of the composition or the oral dosage form, based on the total weight of the composition or the oral dosage form. In one aspect, a composition or an oral dosage form as described herein comprises the glidant from about 0.1% to about 1% by weight of the composition or the oral dosage form, based on the total weight of the composition or the oral dosage form. In one aspect, a composition or an oral dosage form as described herein comprises the glidant at about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, or about 5% by weight of the composition or the oral dosage form, based on the total weight of the composition or the oral dosage form.

In one aspect, a composition or an oral dosage form as described herein comprises the glidant in an intragranular phase from about 0% to about 5% by weight of the composition or the oral dosage form, based on the total weight of the composition or the oral dosage form, and in an extragranular phase from about 0% to about 5% by weight of the composition or the oral dosage form, based on the total weight of the composition or the oral dosage form, wherein the total amount of glidant in the combined intragranular and extragranular phases in the composition or an oral dosage is from about 0.1% to about 5% by weight of the composition or the oral dosage form, based on the total weight of the composition or the oral dosage form.

In one aspect, a composition or an oral dosage form as described herein comprises the glidant in an intragranular phase from about 0% to about 4% by weight of the composition or the oral dosage form, based on the total weight of the composition or the oral dosage form, and in an extragranular phase from about 0% to about 4% by weight of the composition or the oral dosage form, based on the total weight of the composition or the oral dosage form, wherein the total amount of glidant in the combined intragranular and extragranular phases in the composition or an oral dosage is from about 0.1% to about 4% by weight of the composition or the oral dosage form, based on the total weight of the composition or the oral dosage form.

In one aspect, a composition or an oral dosage form as described herein comprises the glidant in an intragranular phase from about 0% to about 3% by weight of the composition or the oral dosage form, based on the total weight of the composition or the oral dosage form, and in an extragranular phase from about 0% to about 3% by weight of the composition or the oral dosage form, based on the total weight of the composition or the oral dosage form, wherein the total amount of glidant in the combined intragranular and extragranular phases in the composition or an oral dosage is from about 0.1% to about 3% by weight of the composition or the oral dosage form, based on the total weight of the composition or the oral dosage form.

In one aspect, a composition or an oral dosage form as described herein comprises the glidant in an intragranular phase from about 0% to about 2% by weight of the composition or the oral dosage form, based on the total weight of the composition or the oral dosage form, and in an extragranular phase from about 0% to about 2% by weight of the composition or the oral dosage form, based on the total weight of the composition or the oral dosage form, wherein the total amount of glidant in the combined intragranular and extragranular phases in the composition or an oral dosage is from about 0.1% to about 2% by weight of the composition or the oral dosage form, based on the total weight of the composition or the oral dosage form.

In one aspect, a composition or an oral dosage form as described herein comprises the glidant in an intragranular phase from about 0.1% to about 2% by weight of the composition or the oral dosage form, based on the total weight of the composition or the oral dosage form, and in an extragranular phase from about 0.1% to about 2% by weight of the composition or the oral dosage form, based on the total weight of the composition or the oral dosage form, wherein the total amount of glidant in the combined intragranular and extragranular phases in the composition or an oral dosage is from about 0.5% to about 2% by weight of the composition or the oral dosage form, based on the total weight of the composition or the oral dosage form.

In one aspect, a composition or an oral dosage form as described herein comprises the glidant in an intragranular phase from about 0% to about 1% by weight of the composition or the oral dosage form, based on the total weight of the composition or the oral dosage form, and in an extragranular phase from about 0% to about 1% by weight of the composition or the oral dosage form, based on the total weight of the composition or the oral dosage form, wherein the total amount of glidant in the combined intragranular and extragranular phases in the composition or an oral dosage is from about 0.1% to about 1% by weight of the composition or the oral dosage form, based on the total weight of the composition or the oral dosage form.

In one aspect, a composition or an oral dosage form as described herein comprises the glidant in an intragranular phase from about 0.25% to about 0.75% by weight of the composition or the oral dosage form, based on the total weight of the composition or the oral dosage form, and in an extragranular phase from about 0.25% to about 0.75% by weight of the composition or the oral dosage form, based on the total weight of the composition or the oral dosage form, wherein the total amount of glidant in the combined intragranular and extragranular phases in the composition or an oral dosage is from about 0.5% to about 1% by weight of the composition or the oral dosage form, based on the total weight of the composition or the oral dosage form.

In one aspect, a composition or an oral dosage form as described herein comprises the glidant in the intragranular phase from about 0% to about 5% by weight of the composition or the oral dosage form, based on the total weight of the composition or the oral dosage form. In one aspect, a composition or an oral dosage form as described herein comprises the glidant in the intragranular phase from about 0% to about 4% by weight of the composition or the oral dosage form, based on the total weight of the composition or the oral dosage form. In one aspect, a composition or an oral dosage form as described herein comprises the glidant in the intragranular phase from about 0% to about 3% by weight of the composition or the oral dosage form, based on the total weight of the composition or the oral dosage form. In one aspect, a composition or an oral dosage form as described herein comprises the glidant in the intragranular phase from about 0% to about 2% by weight of the composition or the oral dosage form, based on the total weight of the composition or the oral dosage form. In one aspect, a composition or an oral dosage form as described herein comprises the glidant in the intragranular phase from about 0% to about 1% by weight of the composition or the oral dosage form, based on the total weight of the composition or the oral dosage form. In one aspect, a composition or an oral dosage form as described herein comprises the glidant in the intragranular phase from about 0.1% to about 5% by weight of the composition or the oral dosage form, based on the total weight of the composition or the oral dosage form. In one aspect, a composition or an oral dosage form as described herein comprises the glidant in the intragranular phase from about 0.1% to about 4% by weight of the composition or the oral dosage form, based on the total weight of the composition or the oral dosage form. In one aspect, a composition or an oral dosage form as described herein comprises the glidant in the intragranular phase from about 0.1% to about 3% by weight of the composition or the oral dosage form, based on the total weight of the composition or the oral dosage form. In one aspect, a composition or an oral dosage form as described herein comprises the glidant in the intragranular phase from about 0.1% to about 2% by weight of the composition or the oral dosage form, based on the total weight of the composition or the oral dosage form. In one aspect, a composition or an oral dosage form as described herein comprises the glidant in the intragranular phase from about 0.1% to about 1% by weight of the composition or the oral dosage form, based on the total weight of the composition or the oral dosage form. In one aspect, a composition or an oral dosage form as described herein comprises the glidant in the intragranular phase from about 0.5% to about 1% by weight of the composition or the oral dosage form, based on the total weight of the composition or the oral dosage form. In one aspect, a composition or an oral dosage form as described herein comprises the glidant in the intragranular phase from about 0.25% to about 0.75% by weight of the composition or the oral dosage form, based on the total weight of the composition or the oral dosage form. In one aspect, a composition or an oral dosage form as described herein comprises the glidant in the intragranular phase at about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, or about 5% by weight of the composition or the oral dosage form, based on the total weight of the composition or the oral dosage form.

In one aspect, a composition or an oral dosage form as described herein comprises the glidant in the extragranular phase from about 0% to about 5% by weight of the composition or the oral dosage form, based on the total weight of the composition or the oral dosage form. In one aspect, a composition or an oral dosage form as described herein comprises the glidant in the extragranular phase from about 0% to about 4% by weight of the composition or the oral dosage form, based on the total weight of the composition or the oral dosage form. In one aspect, a composition or an oral dosage form as described herein comprises the glidant in the extragranular phase from about 0% to about 3% by weight of the composition or the oral dosage form, based on the total weight of the composition or the oral dosage form. In one aspect, a composition or an oral dosage form as described herein comprises the glidant in the extragranular phase from about 0% to about 2% by weight of the composition or the oral dosage form, based on the total weight of the composition or the oral dosage form. In one aspect, a composition or an oral dosage form as described herein comprises the glidant in the extragranular phase from about 0% to about 1% by weight of the composition or the oral dosage form, based on the total weight of the composition or the oral dosage form. In one aspect, a composition or an oral dosage form as described herein comprises the glidant in the extragranular phase from about 0.1% to about 5% by weight of the composition or the oral dosage form, based on the total weight of the composition or the oral dosage form. In one aspect, a composition or an oral dosage form as described herein comprises the glidant in the extragranular phase from about 0.1% to about 4% by weight of the composition or the oral dosage form, based on the total weight of the composition or the oral dosage form. In one aspect, a composition or an oral dosage form as described herein comprises the glidant in the extragranular phase from about 0.1% to about 3% by weight of the composition or the oral dosage form, based on the total weight of the composition or the oral dosage form. In one aspect, a composition or an oral dosage form as described herein comprises the glidant in the extragranular phase from about 0.1% to about 2% by weight of the composition or the oral dosage form, based on the total weight of the composition or the oral dosage form. In one aspect, a composition or an oral dosage form as described herein comprises the glidant in the extragranular phase from about 0.1% to about 1% by weight of the composition or the oral dosage form, based on the total weight of the composition or the oral dosage form. In one aspect, a composition or an oral dosage form as described herein comprises the glidant in the extragranular phase from about 0.5% to about 1% by weight of the composition or the oral dosage form, based on the total weight of the composition or the oral dosage form. In one aspect, a composition or an oral dosage form as described herein comprises the glidant in the extragranular phase from about 0.25% to about 0.75% by weight of the composition or the oral dosage form, based on the total weight of the composition or the oral dosage form. In one aspect, a composition or an oral dosage form as described herein comprises the glidant in the extragranular phase at about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, or about 5% by weight of the composition or the oral dosage form, based on the total weight of the composition or the oral dosage form.

In one aspect, the composition or solid dosage form further comprises a lubricant. Suitable lubricants include, either individually or in combination, glyceryl behaptate; stearic acid and salts thereof, including magnesium, calcium and sodium stearates; hydrogenated vegetable oils; colloidal silica; talc; waxes; boric acid; sodium benzoate; sodium acetate; sodium fumarate; sodium chloride; DL-leucine; polyethylene glycol; sodium oleate; sodium lauryl sulfate; glyceryl dibehenate (Compritol 888 ATO); and magnesium lauryl sulfate. Other non-limiting examples of suitable lubricants include talc, calcium stearate, magnesium stearate, solid polyethylene glycols, sodium lauryl sulfate, sodium stearyl fumerate, and mixtures thereof. In a particular aspect, the lubricant is magnesium stearate. Magnesium stearate reduces friction between the equipment and granulated mixture during compression of tablet formulations.

In one aspect, a composition or an oral dosage form as described herein comprises the lubricant from about 0% to about 5% by weight of the composition or the oral dosage form, based on the total weight of the composition or the oral dosage form. In one aspect, a composition or an oral dosage form as described herein comprises the lubricant from about 0% to about 3% by weight of the composition or the oral dosage form, based on the total weight of the composition or the oral dosage form. In one aspect, a composition or an oral dosage form as described herein comprises the lubricant from about 0% to about 2.5% by weight of the composition or the oral dosage form, based on the total weight of the composition or the oral dosage form. In one aspect, a composition or an oral dosage form as described herein comprises the lubricant from about 0% to about 2% by weight of the composition or the oral dosage form, based on the total weight of the composition or the oral dosage form. In one aspect, a composition or an oral dosage form as described herein comprises the lubricant from about 0.5% to about 2% by weight of the composition or the oral dosage form, based on the total weight of the composition or the oral dosage form. In one aspect, a composition or an oral dosage form as described herein comprises the lubricant from about 0% to about 1.5% by weight of the composition or the oral dosage form, based on the total weight of the composition or the oral dosage form. In one aspect, a composition or an oral dosage form as described herein comprises the lubricant from about 0% to about 1% by weight of the composition or the oral dosage form, based on the total weight of the composition or the oral dosage form. In one aspect, a composition or an oral dosage form as described herein comprises the lubricant at about 0.1%, about 0.2%, about 0.3%, about 0.4%, about 0.5%, about 0.6%, about 0.7%, about 0.8%, about 0.9%, about 1%, about 1.5%, about 2%, about 2.5%, or about 3% by weight of the composition or the oral dosage form, based on the total weight of the composition or the oral dosage form.

In one aspect, a composition or an oral dosage form as described herein comprises the lubricant in an intragranular phase from about 0% to about 5% by weight of the composition or the oral dosage form, based on the total weight of the composition or the oral dosage form, and in an extragranular phase from about 0% to about 5% by weight of the composition or the oral dosage form, based on the total weight of the composition or the oral dosage form, wherein the total amount of lubricant in the combined intragranular and extragranular phases in the composition or an oral dosage is from about 0.1% to about 5% by weight of the composition or the oral dosage form, based on the total weight of the composition or the oral dosage form.

In one aspect, a composition or an oral dosage form as described herein comprises the lubricant in an intragranular phase from about 0% to about 3% by weight of the composition or the oral dosage form, based on the total weight of the composition or the oral dosage form, and in an extragranular phase from about 0% to about 3% by weight of the composition or the oral dosage form, based on the total weight of the composition or the oral dosage form, wherein the total amount of lubricant in the combined intragranular and extragranular phases in the composition or an oral dosage is from about 0.1% to about 3% by weight of the composition or the oral dosage form, based on the total weight of the composition or the oral dosage form.

In one aspect, a composition or an oral dosage form as described herein comprises the lubricant in an intragranular phase from about 0% to about 2.5% by weight of the composition or the oral dosage form, based on the total weight of the composition or the oral dosage form, and in an extragranular phase from about 0% to about 2.5% by weight of the composition or the oral dosage form, based on the total weight of the composition or the oral dosage form, wherein the total amount of lubricant in the combined intragranular and extragranular phases in the composition or an oral dosage is from about 0.1% to about 2.5% by weight of the composition or the oral dosage form, based on the total weight of the composition or the oral dosage form.

In one aspect, a composition or an oral dosage form as described herein comprises the lubricant in an intragranular phase from about 0% to about 2% by weight of the composition or the oral dosage form, based on the total weight of the composition or the oral dosage form, and in an extragranular phase from about 0% to about 2% by weight of the composition or the oral dosage form, based on the total weight of the composition or the oral dosage form, wherein the total amount of lubricant in the combined intragranular and extragranular phases in the composition or an oral dosage is from about 0.1% to about 2% by weight of the composition or the oral dosage form, based on the total weight of the composition or the oral dosage form.

In one aspect, a composition or an oral dosage form as described herein comprises the lubricant in an intragranular phase from about 0.1% to about 2% by weight of the composition or the oral dosage form, based on the total weight of the composition or the oral dosage form, and in an extragranular phase from about 0.1% to about 2% by weight of the composition or the oral dosage form, based on the total weight of the composition or the oral dosage form, wherein the total amount of lubricant in the combined intragranular and extragranular phases in the composition or an oral dosage is from about 0.5% to about 2% by weight of the composition or the oral dosage form, based on the total weight of the composition or the oral dosage form.

In one aspect, a composition or an oral dosage form as described herein comprises the lubricant in an intragranular phase from about 0% to about 1.5% by weight of the composition or the oral dosage form, based on the total weight of the composition or the oral dosage form, and in an extragranular phase from about 0% to about 1.5% by weight of the composition or the oral dosage form, based on the total weight of the composition or the oral dosage form, wherein the total amount of lubricant in the combined intragranular and extragranular phases in the composition or an oral dosage is from about 0.1% to about 1.5% by weight of the composition or the oral dosage form, based on the total weight of the composition or the oral dosage form.

In one aspect, a composition or an oral dosage form as described herein comprises the lubricant in an intragranular phase from about 0% to about 1% by weight of the composition or the oral dosage form, based on the total weight of the composition or the oral dosage form, and in an extragranular phase from about 0% to about 1% by weight of the composition or the oral dosage form, based on the total weight of the composition or the oral dosage form, wherein the total amount of lubricant in the combined intragranular and extragranular phases in the composition or an oral dosage is from about 0.1% to about 1% by weight of the composition or the oral dosage form, based on the total weight of the composition or the oral dosage form.

In one aspect, a composition or an oral dosage form as described herein comprises the lubricant in an intragranular phase from about 0.25% to about 0.75% by weight of the composition or the oral dosage form, based on the total weight of the composition or the oral dosage form, and in an extragranular phase from about 0.25% to about 0.75% by weight of the composition or the oral dosage form, based on the total weight of the composition or the oral dosage form, wherein the total amount of lubricant in the combined intragranular and extragranular phases in the composition or an oral dosage is from about 0.5% to about 1.5% by weight of the composition or the oral dosage form, based on the total weight of the composition or the oral dosage form.

In one aspect, a composition or an oral dosage form as described herein comprises the lubricant in the intragranular phase from about 0% to about 5% by weight of the composition or the oral dosage form, based on the total weight of the composition or the oral dosage form. In one aspect, a composition or an oral dosage form as described herein comprises the lubricant in the intragranular phase from about 0% to about 3% by weight of the composition or the oral dosage form, based on the total weight of the composition or the oral dosage form. In one aspect, a composition or an oral dosage form as described herein comprises the lubricant in the intragranular phase from about 0% to about 2.5% by weight of the composition or the oral dosage form, based on the total weight of the composition or the oral dosage form. In one aspect, a composition or an oral dosage form as described herein comprises the lubricant in the intragranular phase from about 0% to about 2% by weight of the composition or the oral dosage form, based on the total weight of the composition or the oral dosage form. In one aspect, a composition or an oral dosage form as described herein comprises the lubricant in the intragranular phase from about 0% to about 1.5% by weight of the composition or the oral dosage form, based on the total weight of the composition or the oral dosage form. In one aspect, a composition or an oral dosage form as described herein comprises the lubricant in the intragranular phase from about 0% to about 1% by weight of the composition or the oral dosage form, based on the total weight of the composition or the oral dosage form. In one aspect, a composition or an oral dosage form as described herein comprises the lubricant in the intragranular phase from about 0.1% to about 3% by weight of the composition or the oral dosage form, based on the total weight of the composition or the oral dosage form. In one aspect, a composition or an oral dosage form as described herein comprises the lubricant in the intragranular phase from about 0.1% to about 2.5% by weight of the composition or the oral dosage form, based on the total weight of the composition or the oral dosage form. In one aspect, a composition or an oral dosage form as described herein comprises the lubricant in the intragranular phase from about 0.1% to about 2% by weight of the composition or the oral dosage form, based on the total weight of the composition or the oral dosage form. In one aspect, a composition or an oral dosage form as described herein comprises the lubricant in the intragranular phase from about 0.1% to about 1.5% by weight of the composition or the oral dosage form, based on the total weight of the composition or the oral dosage form. In one aspect, a composition or an oral dosage form as described herein comprises the lubricant in the intragranular phase from about 0.1% to about 1% by weight of the composition or the oral dosage form, based on the total weight of the composition or the oral dosage form. In one aspect, a composition or an oral dosage form as described herein comprises the lubricant in the intragranular phase from about 0.5% to about 1% by weight of the composition or the oral dosage form, based on the total weight of the composition or the oral dosage form. In one aspect, a composition or an oral dosage form as described herein comprises the lubricant in the intragranular phase from about 0.25% to about 0.75% by weight of the composition or the oral dosage form, based on the total weight of the composition or the oral dosage form. In one aspect, a composition or an oral dosage form as described herein comprises the lubricant in the intragranular phase at about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, or about 5% by weight of the composition or the oral dosage form, based on the total weight of the composition or the oral dosage form.

In one aspect, a composition or an oral dosage form as described herein comprises the lubricant in the extragranular phase from about 0% to about 5% by weight of the composition or the oral dosage form, based on the total weight of the composition or the oral dosage form. In one aspect, a composition or an oral dosage form as described herein comprises the lubricant in the extragranular phase from about 0% to about 3% by weight of the composition or the oral dosage form, based on the total weight of the composition or the oral dosage form. In one aspect, a composition or an oral dosage form as described herein comprises the lubricant in the extragranular phase from about 0% to about 2.5% by weight of the composition or the oral dosage form, based on the total weight of the composition or the oral dosage form. In one aspect, a composition or an oral dosage form as described herein comprises the lubricant in the extragranular phase from about 0% to about 2% by weight of the composition or the oral dosage form, based on the total weight of the composition or the oral dosage form. In one aspect, a composition or an oral dosage form as described herein comprises the lubricant in the extragranular phase from about 0% to about 1.5% by weight of the composition or the oral dosage form, based on the total weight of the composition or the oral dosage form. In one aspect, a composition or an oral dosage form as described herein comprises the lubricant in the extragranular phase from about 0% to about 1% by weight of the composition or the oral dosage form, based on the total weight of the composition or the oral dosage form. In one aspect, a composition or an oral dosage form as described herein comprises the lubricant in the extragranular phase from about 0.1% to about 3% by weight of the composition or the oral dosage form, based on the total weight of the composition or the oral dosage form. In one aspect, a composition or an oral dosage form as described herein comprises the lubricant in the extragranular phase from about 0.1% to about 2.5% by weight of the composition or the oral dosage form, based on the total weight of the composition or the oral dosage form. In one aspect, a composition or an oral dosage form as described herein comprises the lubricant in the extragranular phase from about 0.1% to about 2% by weight of the composition or the oral dosage form, based on the total weight of the composition or the oral dosage form. In one aspect, a composition or an oral dosage form as described herein comprises the lubricant in the extragranular phase from about 0.1% to about 1.5% by weight of the composition or the oral dosage form, based on the total weight of the composition or the oral dosage form. In one aspect, a composition or an oral dosage form as described herein comprises the lubricant in the extragranular phase from about 0.1% to about 1% by weight of the composition or the oral dosage form, based on the total weight of the composition or the oral dosage form. In one aspect, a composition or an oral dosage form as described herein comprises the lubricant in the extragranular phase from about 0.5% to about 1% by weight of the composition or the oral dosage form, based on the total weight of the composition or the oral dosage form. In one aspect, a composition or an oral dosage form as described herein comprises the lubricant in the extragranular phase from about 0.25% to about 0.75% by weight of the composition or the oral dosage form, based on the total weight of the composition or the oral dosage form. In one aspect, a composition or an oral dosage form as described herein comprises the lubricant in the extragranular phase at about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, or about 5% by weight of the composition or the oral dosage form, based on the total weight of the composition or the oral dosage form.

In one aspect, the composition further comprises one or more fillers (also referred to as a diluent). Suitable fillers, alone or in combination, may be selected from lactose, including anhydrous lactose or lactose monohydrate; starches, including directly compressible and hydrolyzed starches; mannitol, including directly compressible, spray dried and crystalline mannitols; sorbitol; xylitol; dextrose and dextrose monohydrate; sucrose-based diluents including confectioner's sugar; calcium-based diluents including monobasic calcium sulfate monohydrate, dibasic calcium phosphate anhydrous, dibasic calcium phosphate dihydrate; calcium sulfate dihydrate, or granular calcium lactate trihydrate; dextrans; inositol; hydrolyzed cereal solids; amylose; celluloses including food grade sources of amorphous cellulose and powdered cellulose: microcrystalline cellulose, modified or co-processed microcrystalline cellulose, extragranular microcrystalline cellulose, or silicified microcrystalline cellulose; calcium carbonate; glycine; bentonite; polyvinylpyrrolidone; and the like. Other examples of suitable fillers are starch (e.g. cellulose, potato or corn starch), salts (e.g., calcium hydrogenphosphate, magnesium oxide), sugars like lactose (e.g., lactose monohydrate), silicates (e.g., silicium dioxide), talc, isomalt, or polyvinyl alcohol. In a specific aspect, the composition comprises two fillers selected from mannitol and microcrystalline cellulose (e.g., Avicel® PH 101, Avicel® PH 102).

The filler(s) selected preferably exhibit suitable flow properties and, where tablets are desired, improve compressibility. Mixtures of fillers can be used to optimize the desired properties. For example, materials usually compress by either plastic deformation or brittle fracture. Mannitol is a brittle fracturing filler, which in combination with a plastic deforming filler such as microcrystalline cellulose results in an optimal compacting formulation that holds its shape after compaction and have little relaxation/friability. Microcrystalline cellulose (e.g., Avicel® PH 102) was selected as the plastic deforming filler.

In one aspect, a composition or an oral dosage form as described herein comprises the one or more fillers from about 0% to about 90% by weight of the composition or the oral dosage form, based on the total weight of the composition or the oral dosage form. In one aspect, a composition or an oral dosage form as described herein comprises the one or more fillers from about 5% to about 90% by weight of the composition or the oral dosage form, based on the total weight of the composition or the oral dosage form. In one aspect, a composition or an oral dosage form as described herein comprises the one or more fillers from about 10% to about 80% by weight of the composition or the oral dosage form, based on the total weight of the composition or the oral dosage form. In one aspect, a composition or an oral dosage form as described herein comprises the one or more fillers from about 20% to about 70% by weight of the composition or the oral dosage form, based on the total weight of the composition or the oral dosage form. In one aspect, a composition or an oral dosage form as described herein comprises the one or more fillers from about 30% to about 60% by weight of the composition or the oral dosage form, based on the total weight of the composition or the oral dosage form. In one aspect, a composition or an oral dosage form as described herein comprises the one or more fillers from about 40% to about 50% by weight of the composition or the oral dosage form, based on the total weight of the composition or the oral dosage form. In one aspect, a composition or an oral dosage form as described herein comprises the one or more fillers from about 5% to about 15% by weight of the composition or the oral dosage form, based on the total weight of the composition or the oral dosage form. In one aspect, a composition or an oral dosage form as described herein comprises the one or more fillers from about 10% to about 20% by weight of the composition or the oral dosage form, based on the total weight of the composition or the oral dosage form. In one aspect, a composition or an oral dosage form as described herein comprises the one or more fillers from about 15% to about 25% by weight of the composition or the oral dosage form, based on the total weight of the composition or the oral dosage form. In one aspect, a composition or an oral dosage form as described herein comprises the one or more fillers from about 20% to about 30% by weight of the composition or the oral dosage form, based on the total weight of the composition or the oral dosage form. In one aspect, a composition or an oral dosage form as described herein comprises the one or more fillers from about 25% to about 35% by weight of the composition or the oral dosage form, based on the total weight of the composition or the oral dosage form. In one aspect, a composition or an oral dosage form as described herein comprises the one or more fillers from about 30% to about 50% by weight of the composition or the oral dosage form, based on the total weight of the composition or the oral dosage form. In one aspect, a composition or an oral dosage form as described herein comprises the one or more fillers from about 30% to about 40% by weight of the composition or the oral dosage form, based on the total weight of the composition or the oral dosage form. In one aspect, a composition or an oral dosage form as described herein comprises the one or more fillers from about 35% to about 45% by weight of the composition or the oral dosage form, based on the total weight of the composition or the oral dosage form. In one aspect, a composition or an oral dosage form as described herein comprises the one or more fillers from about 45% to about 55% by weight of the composition or the oral dosage form, based on the total weight of the composition or the oral dosage form. In one aspect, a composition or an oral dosage form as described herein comprises the one or more fillers from about 55% to about 65% by weight of the composition or the oral dosage form, based on the total weight of the composition or the oral dosage form. In one aspect, a composition or an oral dosage form as described herein comprises the one or more fillers from about 65% to about 75% by weight of the composition or the oral dosage form, based on the total weight of the composition or the oral dosage form. In one aspect, a composition or an oral dosage form as described herein comprises the one or more fillers from about 75% to about 85% by weight of the composition or the oral dosage form, based on the total weight of the composition or the oral dosage form. In one aspect, a composition or an oral dosage form as described herein comprises the one or more fillers from about 50% to about 60% by weight of the composition or the oral dosage form, based on the total weight of the composition or the oral dosage form. In one aspect, a composition or an oral dosage form as described herein comprises the one or more fillers from about 60% to about 70% by weight of the composition or the oral dosage form, based on the total weight of the composition or the oral dosage form. In one aspect, a composition or an oral dosage form as described herein comprises the one or more fillers from about 70% to about 80% by weight of the composition or the oral dosage form, based on the total weight of the composition or the oral dosage form. In one aspect, a composition or an oral dosage form as described herein comprises the one or more fillers from about 80% to about 90% by weight of the composition or the oral dosage form, based on the total weight of the composition or the oral dosage form. In one aspect, a composition or an oral dosage form as described herein comprises the one or more fillers at about 5%, about 10%, about 15%, about 20%, about 25%, about 30%, about 35%, about 45%, about 50%, about 55%, about 60%, about 65%, about 70%, about 75%, about 80%, about 85%, or about 90% by weight of the composition or the oral dosage form, based on the total weight of the composition or the oral dosage form.

In one aspect, a composition or an oral dosage form as described herein comprises the one or more fillers in the intragranular phase from about 0% to about 90% (e.g., about 20% to about 60%) by weight of the composition or the oral dosage form, based on the total weight of the composition or the oral dosage form, and in the extragranular phase from about 0% to about 90% (e.g., about 0% to about 20%) by weight of the composition or the oral dosage form, based on the total weight of the composition or the oral dosage form, wherein the total amount of the one or more fillers in the combined intragranular and extragranular phases in the composition or an oral dosage is from about 5% to about 90% (e.g., about 20% to about 60%) by weight of the composition or the oral dosage form, based on the total weight of the composition or the oral dosage form.

In one aspect, a composition or an oral dosage form as described herein comprises the one or more fillers in the intragranular phase from about 0% to about 80% by weight of the composition or the oral dosage form, based on the total weight of the composition or the oral dosage form, and in the extragranular phase from about 0% to about 80% by weight of the composition or the oral dosage form, based on the total weight of the composition or the oral dosage form, wherein the total amount of the one or more fillers in the combined intragranular and extragranular phases in the composition or an oral dosage is from about 5% to about 80% by weight of the composition or the oral dosage form, based on the total weight of the composition or the oral dosage form.

In one aspect, a composition or an oral dosage form as described herein comprises the one or more fillers in the intragranular phase from about 0% to about 70% by weight of the composition or the oral dosage form, based on the total weight of the composition or the oral dosage form, and in the extragranular phase from about 0% to about 70% by weight of the composition or the oral dosage form, based on the total weight of the composition or the oral dosage form, wherein the total amount of the one or more fillers in the combined intragranular and extragranular phases in the composition or an oral dosage is from about 5% to about 70% by weight of the composition or the oral dosage form, based on the total weight of the composition or the oral dosage form.

In one aspect, a composition or an oral dosage form as described herein comprises the one or more fillers in the intragranular phase from about 0% to about 60% by weight of the composition or the oral dosage form, based on the total weight of the composition or the oral dosage form, and in the extragranular phase from about 0% to about 60% by weight of the composition or the oral dosage form, based on the total weight of the composition or the oral dosage form, wherein the total amount of the one or more fillers in the combined intragranular and extragranular phases in the composition or an oral dosage is from about 5% to about 60% by weight of the composition or the oral dosage form, based on the total weight of the composition or the oral dosage form.

In one aspect, a composition or an oral dosage form as described herein comprises the one or more fillers in the intragranular phase from about 0% to about 50% by weight of the composition or the oral dosage form, based on the total weight of the composition or the oral dosage form, and in the extragranular phase from about 0% to about 50% by weight of the composition or the oral dosage form, based on the total weight of the composition or the oral dosage form, wherein the total amount of the one or more fillers in the combined intragranular and extragranular phases in the composition or an oral dosage is from about 5% to about 50% by weight of the composition or the oral dosage form, based on the total weight of the composition or the oral dosage form.

In one aspect, a composition or an oral dosage form as described herein comprises the one or more fillers in the intragranular phase from about 30% to about 50% by weight of the composition or the oral dosage form, based on the total weight of the composition or the oral dosage form, and in the extragranular phase from about 0% to about 10% by weight of the composition or the oral dosage form, based on the total weight of the composition or the oral dosage form, wherein the total amount of the one or more fillers in the combined intragranular and extragranular phases in the composition or an oral dosage is from about 30% to about 60% by weight of the composition or the oral dosage form, based on the total weight of the composition or the oral dosage form.

In one aspect, a composition or an oral dosage form as described herein comprises the one or more fillers in the intragranular phase from about 30% to about 45% by weight of the composition or the oral dosage form, based on the total weight of the composition or the oral dosage form, and in the extragranular phase from about 1% to about 10% by weight of the composition or the oral dosage form, based on the total weight of the composition or the oral dosage form, wherein the total amount of the one or more fillers in the combined intragranular and extragranular phases in the composition or an oral dosage is from about 31% to about 50% by weight of the composition or the oral dosage form, based on the total weight of the composition or the oral dosage form.

In one aspect, a composition or an oral dosage form as described herein comprises the one or more fillers in the intragranular phase from about 0% to about 40% by weight of the composition or the oral dosage form, based on the total weight of the composition or the oral dosage form, and in the extragranular phase from about 0% to about 40% by weight of the composition or the oral dosage form, based on the total weight of the composition or the oral dosage form, wherein the total amount of the one or more fillers in the combined intragranular and extragranular phases in the composition or an oral dosage is from about 5% to about 40% by weight of the composition or the oral dosage form, based on the total weight of the composition or the oral dosage form.

In one aspect, a composition or an oral dosage form as described herein comprises the one or more fillers in the intragranular phase from about 0% to about 30% by weight of the composition or the oral dosage form, based on the total weight of the composition or the oral dosage form, and in the extragranular phase from about 0% to about 30% by weight of the composition or the oral dosage form, based on the total weight of the composition or the oral dosage form, wherein the total amount of the one or more fillers in the combined intragranular and extragranular phases in the composition or an oral dosage is from about 5% to about 30% by weight of the composition or the oral dosage form, based on the total weight of the composition or the oral dosage form.

In one aspect, a composition or an oral dosage form as described herein comprises the one or more fillers in the intragranular phase from about 0% to about 20% by weight of the composition or the oral dosage form, based on the total weight of the composition or the oral dosage form, and in an extragranular phase from about 0% to about 20% by weight of the composition or the oral dosage form, based on the total weight of the composition or the oral dosage form, wherein the total amount of the one or more fillers in the combined intragranular and extragranular phases in the composition or an oral dosage is from about 5% to about 20% by weight of the composition or the oral dosage form, based on the total weight of the composition or the oral dosage form.

In one aspect, a composition or an oral dosage form as described herein comprises the one or more fillers in the intragranular phase from about 0% to about 10% by weight of the composition or the oral dosage form, based on the total weight of the composition or the oral dosage form, and in the extragranular phase from about 0% to about 10% by weight of the composition or the oral dosage form, based on the total weight of the composition or the oral dosage form, wherein the total amount of the one or more fillers in the combined intragranular and extragranular phases in the composition or an oral dosage is from about 5% to about 10% by weight of the composition or the oral dosage form, based on the total weight of the composition or the oral dosage form.

In one aspect, a composition or an oral dosage form as described herein comprises the one or more filler in the intragranular phase from about 0% to about 90% by weight of the composition or the oral dosage form, based on the total weight of the composition or the oral dosage form. In one aspect, a composition or an oral dosage form as described herein comprises the one or more filler in the intragranular phase from about 0% to about 80% by weight of the composition or the oral dosage form, based on the total weight of the composition or the oral dosage form. In one aspect, a composition or an oral dosage form as described herein comprises the one or more filler in the intragranular phase from about 0% to about 70% by weight of the composition or the oral dosage form, based on the total weight of the composition or the oral dosage form. In one aspect, a composition or an oral dosage form as described herein comprises the one or more filler in the intragranular phase from about 0% to about 60% by weight of the composition or the oral dosage form, based on the total weight of the composition or the oral dosage form. In one aspect, a composition or an oral dosage form as described herein comprises the one or more filler in the intragranular phase from about 0% to about 50% by weight of the composition or the oral dosage form, based on the total weight of the composition or the oral dosage form. In one aspect, a composition or an oral dosage form as described herein comprises the one or more filler in the intragranular phase from about 10% to about 50% by weight of the composition or the oral dosage form, based on the total weight of the composition or the oral dosage form. In one aspect, a composition or an oral dosage form as described herein comprises the one or more filler in the intragranular phase from about 20% to about 50% by weight of the composition or the oral dosage form, based on the total weight of the composition or the oral dosage form. In one aspect, a composition or an oral dosage form as described herein comprises the one or more filler in the intragranular phase from about 30% to about 50% by weight of the composition or the oral dosage form, based on the total weight of the composition or the oral dosage form. In one aspect, a composition or an oral dosage form as described herein comprises the one or more filler in the intragranular phase from about 35% to about 45% by weight of the composition or the oral dosage form, based on the total weight of the composition or the oral dosage form. In one aspect, a composition or an oral dosage form as described herein comprises the one or more filler in the intragranular phase from about 0% to about 40% by weight of the composition or the oral dosage form, based on the total weight of the composition or the oral dosage form. In one aspect, a composition or an oral dosage form as described herein comprises the one or more filler in the intragranular phase from about 30% to about 40% by weight of the composition or the oral dosage form, based on the total weight of the composition or the oral dosage form. In one aspect, a composition or an oral dosage form as described herein comprises the one or more filler in the intragranular phase from about 0% to about 30% by weight of the composition or the oral dosage form, based on the total weight of the composition or the oral dosage form. In one aspect, a composition or an oral dosage form as described herein comprises the one or more filler in the intragranular phase from about 0% to about 20% by weight of the composition or the oral dosage form, based on the total weight of the composition or the oral dosage form. In one aspect, a composition or an oral dosage form as described herein comprises the one or more filler in the intragranular phase from about 0% to about 10% by weight of the composition or the oral dosage form, based on the total weight of the composition or the oral dosage form. In one aspect, a composition or an oral dosage form as described herein comprises the one or more fillers in the intragranular phase at about 10%, about 15%, about 20%, about 25%, about 30%, about 35%, about 40%, about 45%, about 50%, about 55%, about 60%, about 65%, about 70%, about 75%, about 80%, about 85%, or about 90% by weight of the composition or the oral dosage form, based on the total weight of the composition or the oral dosage form.

In one aspect, a composition or an oral dosage form as described herein comprises the one or more filler in the extragranular phase from about 0% to about 90% by weight of the composition or the oral dosage form, based on the total weight of the composition or the oral dosage form. In one aspect, a composition or an oral dosage form as described herein comprises the one or more filler in the extragranular phase from about 0% to about 80% by weight of the composition or the oral dosage form, based on the total weight of the composition or the oral dosage form. In one aspect, a composition or an oral dosage form as described herein comprises the one or more filler in the extragranular phase from about 0% to about 70% by weight of the composition or the oral dosage form, based on the total weight of the composition or the oral dosage form. In one aspect, a composition or an oral dosage form as described herein comprises the one or more filler in the extragranular phase from about 0% to about 60% by weight of the composition or the oral dosage form, based on the total weight of the composition or the oral dosage form. In one aspect, a composition or an oral dosage form as described herein comprises the one or more filler in the extragranular phase from about 0% to about 50% by weight of the composition or the oral dosage form, based on the total weight of the composition or the oral dosage form. In one aspect, a composition or an oral dosage form as described herein comprises the one or more filler in the extragranular phase from about 0% to about 40% by weight of the composition or the oral dosage form, based on the total weight of the composition or the oral dosage form. In one aspect, a composition or an oral dosage form as described herein comprises the one or more filler in the extragranular phase from about 0% to about 30% by weight of the composition or the oral dosage form, based on the total weight of the composition or the oral dosage form. In one aspect, a composition or an oral dosage form as described herein comprises the one or more filler in the extragranular phase from about 0% to about 20% by weight of the composition or the oral dosage form, based on the total weight of the composition or the oral dosage form. In one aspect, a composition or an oral dosage form as described herein comprises the one or more filler in the extragranular phase from about 0% to about 10% by weight of the composition or the oral dosage form, based on the total weight of the composition or the oral dosage form. In one aspect, a composition or an oral dosage form as described herein comprises the one or more filler in the extragranular phase from about 0.5% to about 10% by weight of the composition or the oral dosage form, based on the total weight of the composition or the oral dosage form. In one aspect, a composition or an oral dosage form as described herein comprises the one or more filler in the extragranular phase from about 0.5% to about 5% by weight of the composition or the oral dosage form, based on the total weight of the composition or the oral dosage form. In one aspect, a composition or an oral dosage form as described herein comprises the one or more filler in the extragranular phase from about 1% to about 5% by weight of the composition or the oral dosage form, based on the total weight of the composition or the oral dosage form. In one aspect, a composition or an oral dosage form as described herein comprises the one or more filler in the extragranular phase at about 10%, about 15%, about 20%, about 25%, about 30%, about 35%, about 40%, about 45%, about 50%, about 55%, about 60%, about 65%, about 70%, about 75%, about 80%, about 85%, or about 90% by weight of the composition or the oral dosage form, based on the total weight of the composition or the oral dosage form.

In one aspect, a composition or an oral dosage form as described herein comprises one filler. In one aspect, a composition or an oral dosage form as described herein comprises two fillers. In one aspect, one filler is in the intragranular phase and the second filler is in the extragranular phase. In one aspect, one filler is in the both the intragranular phase and the extragranular phase and the second filler is only in the extragranular phase. In one aspect, one filler is in the both the intragranular phase and the extragranular phase and the second filler is only in the intragranular phase. In one aspect, both fillers are both in the intragranular phase and the extragranular phases.

In one aspect, in a composition or an oral dosage form as described herein, the intragranular phase comprises two fillers selected from mannitol and microcrystalline cellulose; and the extragranular phase comprises the filler selected from mannitol.

In one aspect, in a composition or an oral dosage form as described herein, both the intragranular phase and extragranular phase comprise both two fillers selected from mannitol and microcrystalline cellulose.

In one aspect, a composition or an oral dosage form as described herein comprises two fillers, wherein the first filler is from about 5% to about 30% by weight of the composition or the oral dosage form, based on the total weight of the composition or the oral dosage form, and the second filler is from about 10% to about 30% by weight of the composition or the oral dosage form, based on the total weight of the composition or the oral dosage form.

In one aspect, a composition or an oral dosage form as described herein comprises two fillers, wherein the first filler is from about 15% to about 30% by weight of the composition or the oral dosage form, based on the total weight of the composition or the oral dosage form, and the second filler is from about 15% to about 25% by weight of the composition or the oral dosage form, based on the total weight of the composition or the oral dosage form.

In one aspect, a composition or an oral dosage form as described herein further comprises a non-functional polymer coating. A “nonfunctional film coating” is used to change tablet appearance, swallowability, mask the taste of the active pharmaceutical ingredient (API), and to protect tablets from the negative environmental effects such as humidity, oxidation, and light effects and potentially improve drug product stability. It will not however alter (extend or delay) the release rate of the drug molecule from the tablets. The suitable non-functional polymer coatings, alone or in combination, may be selected from hydroxypropyl methylcellulose, hydroxypropyl cellulose, hydroxyethyl cellulose, methyl cellulose, sodium carboxymethyl cellulose, polyvinyl pyrrolidone, polyvinyl pyrrolidone-polyvinyl acetate copolymer, polyvinyl alcohol, polyvinyl alcohol-polyethylene glycol copolymers, acrylic polymers, and polyethylene glycols. In some embodiments, the non-functional polymer coating is selected from macrogol (PEG) PVA graft copolymer, and polyvinyl alcohol. In a specific embodiment, the non-functional polymer coating is polyvinyl alcohol.

In one aspect, a composition or an oral dosage form as described herein further comprises the non-functional coating polymer from about 0% to about 10% (e.g., about 0.5% to about 6% or about 2% to about 5%) by weight of the composition, based on the total weight of the composition.

In one aspect, a composition or an oral dosage form as described herein further comprises a non-functional polymer coating, wherein the non-functional polymer coating includes a pigment. In some embodiments, the pigment is an iron oxide-based pigment.

In one aspect, a composition or an oral dosage form as described herein further comprises a non-functional polymer coating, wherein the non-functional polymer coating does not include a pigment.

In one aspect, a composition or an oral dosage form as described herein comprises two fillers, wherein the first filler is from about 15% to about 25% by weight of the composition or the oral dosage form, based on the total weight of the composition or the oral dosage form, and the second filler is from about 15% to about 25% by weight of the composition or the oral dosage form, based on the total weight of the composition or the oral dosage form.

In one aspect, a composition or an oral dosage form as described herein comprises two fillers, wherein the first filler is from about 10% to about 20% by weight of the composition or the oral dosage form in the intragranular phase and from about 1% to about 10% in the extragranular phase, based on the total weight of the composition or the oral dosage form, and the second filler is from about 10% to about 30% by weight of the composition or the oral dosage form in the intragranular phase, based on the total weight of the composition or the oral dosage form.

In one aspect, a composition or an oral dosage form as described herein comprises two fillers, wherein the first filler is from about 10% to about 30% by weight of the composition or the oral dosage form in the intragranular phase and from about 1% to about 10% in the extragranular phase, based on the total weight of the composition or the oral dosage form, and the second filler is from about 10% to about 30% by weight of the composition or the oral dosage form in the intragranular phase and from about 1% to about 10% in the extragranular phase, based on the total weight of the composition or the oral dosage form.

In one aspect, the composition further comprises a moisture scavenger. Non-limiting examples of suitable moisture scavenger include starch, celluloses, celluloses derivatives, silica and silica derivatives. Specific examples are corn starch, rice starch, cellulose, microcrystalline cellulose, sodium carboxymethyl cellulose, hydroxypropyl cellulose, hydroxypropylmethyl cellulose, mesoporous silica, magnesium aluminum silicate, or a silica including a fumed silicon dioxide. Notably, the microcrystalline cellulose may be Avicel™ and/or the fumed silicon dioxide may be Cabosil™. In one aspect, the moisture scavenger is a starch. In a particular aspect, the starch is pregelatinized starch.

In one aspect, a composition or an oral dosage form as described herein comprises the moisture scavenger from 0% to about 30% by weight of the composition or the oral dosage form, based on the total weight of the composition or the oral dosage form. In another aspect, a composition or an oral dosage form as described herein comprises the moisture scavenger from about 0.5% to about 30% by weight of the composition or the oral dosage form, based on the total weight of the composition or the oral dosage form. In one aspect, a composition or an oral dosage form as described herein comprises the moisture scavenger from about 0.5% to about 5% by weight of the composition or the oral dosage form, based on the total weight of the composition or the oral dosage form. In one aspect, a composition or an oral dosage form as described herein comprises the moisture scavenger from about 5% to about 10% by weight of the composition or the oral dosage form, based on the total weight of the composition or the oral dosage form. In one aspect, a composition or an oral dosage form as described herein comprises the moisture scavenger from about 10% to about 15% by weight of the composition or the oral dosage form, based on the total weight of the composition or the oral dosage form. In one aspect, a composition or an oral dosage form as described herein comprises the moisture scavenger from about 15% to about 20% by weight of the composition or the oral dosage form, based on the total weight of the composition or the oral dosage form. In one aspect, a composition or an oral dosage form as described herein comprises the moisture scavenger from about 20% to about 25% by weight of the composition or the oral dosage form, based on the total weight of the composition or the oral dosage form. In one aspect, a composition or an oral dosage form as described herein comprises the moisture scavenger from about 25% to about 30% by weight of the composition or the oral dosage form, based on the total weight of the composition or the oral dosage form. In one aspect, a composition or an oral dosage form as described herein comprises the moisture scavenger from about 5% to about 15% by weight of the composition or the oral dosage form, based on the total weight of the composition or the oral dosage form. In one aspect, a composition or an oral dosage form as described herein comprises the moisture scavenger from about 2% to about 4% by weight of the composition or the oral dosage form, based on the total weight of the composition or the oral dosage form. In one aspect, a composition or an oral dosage form as described herein comprises the moisture scavenger about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%, by weight of the composition or the oral dosage form, based on the total weight of the composition or the oral dosage form. In an alternative aspect, the composition does not include a moisture scavenger.

As used herein, “the total weight of the oral dosage form” means the material within the oral dosage form (e.g., within a capsule) or the oral dosage form without any coating (e.g., without the tablet coating).

As used herein, the term “about”, when used in connection with amounts, weight percent, or a ratio of ingredients in a composition or a dosage form, includes the value of a specified amount, weight percent or ratio (or a range of the amount, weight percent or ratio) that is recognized by one of ordinary skill in the art to provide a pharmacological effect equivalent to that obtained from the specified amount, or weight percent, or ratio.

An oral dosage form can be prepared into any suitable dosage forms, such as capsule, dragee, granule, powder, or tablet. In a particular aspect, the oral dosage form is a capsule. In one embodiment, the size of the capsule is from size 4 to size 0EL. In one embodiment, the size of the capsule is from size 4 to size 00. In another embodiment, the size of the capsule is from size 4 to size 0. In certain embodiments, the size of the capsule is from size 3 to size 0. In some embodiments, the size of the capsule is 0. In other embodiments, the size of the capsule is 0EL. In other embodiments, the size of the capsule is 00. In certain embodiments, the size of the capsule is 1. In some embodiments, the size of the capsule is 2. In other embodiments, the size of the capsule is 3. In certain embodiments, the size of the capsule is 4. In a particular aspect, the oral dosage form is a tablet. In one embodiment, the oral dosage form is a tablet with a non-functional polymer coating. In one embodiment, the size of the tablet is 6.1 mm round biconvex, the size of tablet is 6.35 mm round biconvex, the size of tablet is 9.0 mm round biconvex, or the size of tablet is 9.5 mm round biconvex. In one embodiment, the size of the tablet is 6.9×16.9 mm oval biconvex, the size of the tablet is 8×16 mm oval biconvex, the size of the tablet is 9×18 mm oval biconvex, the size of tablet is 9.5×18.4 mm oval biconvex, or the size of tablet is 10×19 mm oval biconvex.

In one aspect, the oral dosage form or the composition as described herein comprises about 10 mg, about 20 mg, about 25 mg, about 30 mg, about 40 mg, about 50 mg, about 60 mg, about 70 mg, about 80 mg, about 90 mg, about 100 mg, about 110 mg, 120 mg, 130 mg, 140 mg, 150 mg, or about 200 mg of Compound (I) free base, or an equivalent amount of a pharmaceutically acceptable salt thereof. In some embodiments, the oral dosage form (e.g., tablet) comprises about 50 mg of Compound (I) free base or an equivalent amount of a pharmaceutically acceptable salt thereof.

In some embodiments, the amorphous solid dispersion comprises about 10 mg, about 20 mg, about 25 mg, about 30 mg, about 40 mg, about 50 mg, about 60 mg, about 70 mg, about 80 mg, about 90 mg, about 100 mg, about 125 mg, about 150 mg, about 175 mg, or about 200 mg of Compound (I) free base or an equivalent amount of a pharmaceutically acceptable salt thereof.

In some embodiments, the amorphous solid dispersion comprises about 100 mg to 200 mg of Compound (I) free base or an equivalent amount of a pharmaceutically acceptable salt thereof.

In one aspect, a composition or an oral dosage form as described herein comprises:
an intragranular phase, wherein the intragranular phase comprises: (i) an amorphous solid dispersion comprising Compound (I) or a pharmaceutically acceptable salt thereof, and a pharmaceutically acceptable polymer, and (ii) a surfactant; and
an extragranular phase, wherein the extragranular phase comprises at least one of the following: a surfactant, a disintegrant, a glidant, a lubricant, and a filler;
wherein the intragranular phase is from about 80% to about 95% by weight of the composition or the oral dosage form, based on the total weight of the composition or the oral dosage form, and the extragranular phase is from about 5% to about 20% by weight of the composition or the oral dosage form, based on the total weight of the composition or the oral dosage form.

In one embodiment, the intragranular phase is from about 85% to about 95% by weight of the composition or the oral dosage form, based on the total weight of the composition or the oral dosage form, and the extragranular phase is from about 5% to about 15% by weight of the composition or the oral dosage form, based on the total weight of the composition or the oral dosage form. In one specific embodiment, the oral dosage form is a capsule.

In one embodiment, the intragranular phase is from about 80% to about 90% by weight of the composition or the oral dosage form, based on the total weight of the composition or the oral dosage form, and the extragranular phase is from about 10% to about 20% by weight of the composition or the oral dosage form, based on the total weight of the composition or the oral dosage form. In one specific embodiment, the oral dosage form is a tablet.

In one embodiment, the present disclosure features an oral dosage form comprising (a) an amorphous solid dispersion of Compound (I) free base, or a pharmaceutically acceptable salt thereof; and a polymer; (b) a surfactant; (c) a disintegrant; (d) a glidant; (e) a lubricant; and (f) one or more fillers. In one aspect, the amount of the amorphous solid dispersion is as described above (e.g., about 40% to about 60%). In one aspect, the amount of the surfactant is as described above for a surfactant (e.g., about 0.25% to about 5%). In one aspect, the amount of the disintegrant is as described above for a disintegrant (e.g., about 5.1% to about 10%). In one aspect, the amount of the glidant is as described above for a glidant (e.g., about 0.5% to about 2%). In one aspect, the amount of the lubricant is as described above for a lubricant (e.g., about 0.5% to about 2%). In one aspect, the amount of the filler is as described above for a filler (e.g., about 31% to about 50%).

In one embodiment, the present disclosure features an oral dosage form comprising (a) an amorphous solid dispersion of Compound (I) free base, or a pharmaceutically acceptable salt thereof; and hydroxypropyl methylcellulose acetate succinate (HPMCAS); (b) poloxamer 407; (c) crospovidone; (d) colloidal silicon dioxide; (e) magnesium stearate; (f) microcrystalline cellulose (MCC) and (g) mannitol. In one aspect, the amount of the amorphous solid dispersion is as described above (e.g., about 40% to about 60%). In one aspect, the amount of poloxamer 407 is as described above for a surfactant (e.g., about 0.25% to about 5%). In one aspect, the amount of crospovidone is as described above for a disintegrant (e.g., about 5.1% to about 10%). In one aspect, the amount of colloidal silicon dioxide is as described above for a glidant (e.g., about 0.5% to about 2%). In one aspect, the amount of magnesium stearateis as described above for a lubricant (e.g., about 0.5% to about 2%). In one aspect, the amount of microcrystalline cellulose and mannitol is as described above for a filler (e.g., about 31% to about 50%).

In one aspect, the present disclosure provides an oral dosage form comprising:
a) an amorphous solid dispersion comprising: Compound (I) free base, or an equivalent amount of a pharmaceutically acceptable salt thereof, and hydroxypropyl methylcellulose acetate succinate, wherein the Compound (I) free base or an equivalent amount of a pharmaceutically acceptable salt thereof and the hydroxypropyl methylcellulose acetate succinate are in about a 1:1 weight ratio;
b) a surfactant, and optionally,
c) a filler; disintegrant, glidant, and/or a lubricant.

In one aspect, the present disclosure provides oral dosage form comprising:
a) an amorphous solid dispersion comprising: Compound (I) free base, or an equivalent amount of a pharmaceutically acceptable salt thereof, and hydroxypropyl methylcellulose acetate succinate, wherein the Compound (I) free base or an equivalent amount of a pharmaceutically acceptable salt thereof and the hydroxypropyl methylcellulose acetate succinate are in about a 1:1 weight ratio;
b) a surfactant, wherein the composition comprises an intragranular phase and an extragranular phase, wherein the surfactant is present in the intragranular phase and the extragranular phase;
c) a disintegrant;
d) a glidant;
e) a lubricant; and
f) one or more fillers.

In one aspect, in the oral dosage form disclosed herein, the intragranular phase comprises (a) an amorphous solid dispersion of Compound (I) free base, or a pharmaceutically acceptable salt thereof; and hydroxypropyl methylcellulose acetate succinate (HPMCAS); (b) poloxamer 407; (c) crospovidone; (d) colloidal silicon dioxide; (e) magnesium stearate; and (f) microcrystalline cellulose (MCC) and (g) mannitol; and the extragranular phase comprises (b) poloxamer 407; (c) crospovidone; (d) colloidal silicon dioxide; (e) magnesium stearate; and (g) mannitol.

In one aspect, in the oral dosage form disclosed herein, the intragranular phase comprises (a) an amorphous solid dispersion of Compound (I) free base, or a pharmaceutically acceptable salt thereof; and hydroxypropyl methylcellulose acetate succinate (HPMCAS); (b) poloxamer 407; (c) crospovidone; (d) colloidal silicon dioxide; (e) magnesium stearate; and (f) microcrystalline cellulose (MCC) and (g) mannitol; and the extragranular phase comprises (b) poloxamer 407; (c) crospovidone; (d) colloidal silicon dioxide; (e) magnesium stearate; (f) microcrystalline cellulose (MCC) and (g) mannitol.

In one aspect, in the oral dosage form disclosed herein, the intragranular phase comprises (a) an amorphous solid dispersion of Compound (I) free base, or a pharmaceutically acceptable salt thereof; and hydroxypropyl methylcellulose acetate succinate (HPMCAS); (b) poloxamer 407; (c) crospovidone; (d) colloidal silicon dioxide; (e) magnesium stearate; and (f) microcrystalline cellulose (MCC) and mannitol; and the extragranular phase comprises (b) poloxamer 407; (c) crospovidone; (d) colloidal silicon dioxide; (e) magnesium stearate; and (f) microcrystalline cellulose (MCC) and mannitol; and a non-functional coating comprises of polyvinyl alcohol.

In certain embodiments, the dosage form is a capsule, wherein the capsule disintegrates in less than 15 minutes (e.g., 3 minutes, e.g., about 5 minutes, about 7 minutes, about 10 minutes, about 15 minutes) using USP <701>, wherein the procedure for uncoated or plain-coated tablets is used, wherein the capsule is placed in each of the 6 tubes of the basket (Basket type A) along with the disc and analytical grade water was added, and the temperature was maintained at 37° C.±2° C. In a particular embodiment, the capsule disintegrates in less than 15 minutes.

In some embodiments, the dosage form is a capsule or a tablet, wherein at least 50%, at least 60%, at least 70%, at least 75%, at least 80%, at least 85%, at least 90%, or at least 95%, of the Compound (I) is released in about 30 minutes, using USP II apparatus with a biorelevant media (0.01N HCl; or 2.24 mg/mL FaSSIF in 100 mM PBS buffer, pH 6.8) with a vessel volume of 100 mL, and a paddle speed of 100 rpm±2 rpm, and a temperature of 37.0±0.5° C.

In some embodiments, the dosage form is a capsule, wherein at least 50%, at least 60%, at least 70%, at least 75%, at least 80%, at least 85%, at least 90%, or at least 95%, of the Compound (I) is released in about 30 minutes, the following dissolution protocol in biorelevant media:

| Parameter | Value | |
|---|---|---|
| Apparatus | Distek 2100 C Dissolution Apparatus, USP Type II | |
| Gastric Media | 0.01N HCl | |
| (SGF) | | |
| Gastric Dose | 2000 | μgA/mL |
| Intestinal Media | 2.24 mg/mL FaSSIF in 100 mM PBS buffer, pH 6.8 | |
| (FaSSIF) | | |
| Intestinal Dose | 1000 | μgA/mL |
| Paddle RPM | 100 | |
| Bath Temp | 37.0 ± 0.5° | C. |
| Vessel Volume | 100 | mL |

| | |
|---|---|
| Centrifuged | 3 min @ 13k RPM |
| Replicates | n = 3 vessels |

Another embodiment of the present disclosure features a method for preparing the amorphous solid dispersion described herein, comprising: mixing the Compound (I) free base or an equivalent amount of a pharmaceutically acceptable salt thereof, with the polymer in about a 1:1 ratio; adding one or more solvents and removing the solvent(s) by heating.

### Treatment Methods

Another embodiment of the present disclosure features a method of treating an EGFR-altered cancer comprising administering to a patient in need thereof a therapeutically effective amount of the compositions and oral dosage forms disclosed herein.

Another embodiment of the present disclosure features a method of inhibiting certain mutant forms of epidermal growth factor receptor (EGFR) in a subject in need thereof, comprising administering to a patient in need thereof a therapeutically effective amount of the compositions and oral dosage forms disclosed herein. Mutant forms of EGFR include for example, EGFR with LRTMCS mutation (the exon 19 deletion (dell9) or exon 21 (L858R) substitution mutation, T790M mutation, and C797S mutation). Subjects “in need of inhibiting EGFR” are those having a disease for which a beneficial therapeutic effect can be achieved by inhibiting at least one mutant EGFR, e.g., a slowing in disease progression, alleviation of one or more symptoms associated with the disease or increasing the longevity of the subject in view of the disease.

In some embodiments, the disclosure provides a method of treating a disease/condition/or cancer associated with or modulated by mutant EGFR, wherein the inhibition of the mutant EGFR is of therapeutic benefit, including but not limited to the treatment of cancer in a subject in need thereof. The method comprises administering to the subject a therapeutically effective amount of the compositions and oral dosage forms disclosed herein.

In another embodiment, the disclosure provides a method of treating a subject with cancer, comprising administering to a patient in need thereof a therapeutically effective amount of the compositions and oral dosage forms disclosed herein. Cancers to be treated according to the disclosed methods include lung cancer, colon cancer, urothelial cancer, breast cancer, prostate cancer, brain cancers, ovarian cancer, gastric cancer, pancreatic cancer, head and neck cancer, bladder cancer, and mesothelioma, including metastasis (in particular brain metastasis) of all cancers listed. Typically, the cancer is characterized by at one or more EGFR mutations described herein. In a specific embodiment, the cancer has progressed on or after EGFR tyrosine kinase inhibitor (TKI) Therapy. In a specific embodiment, the disease has progressed on or after first line osimertinib.

In a specific embodiment, the cancer to be treated is lung cancer. In a more specific embodiment, the cancer is non-small cell lung cancer (NSCLC). In some embodiments, the lung cancer is locally advanced or metastatic NSCLC, NSCLC adenocarcinoma, NSCLC with squamous histology and NSCLC with non-squamous histology. In another embodiment, the lung cancer is NSCLC adenocarcinoma. In another specific embodiment, the lung cancer (or non-small cell lung cancer) has metastasized to the brain.

The precise amount of compound administered to provide an “therapeutically effective amount” to the subject will depend on the mode of administration, the type, and severity of the cancer, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-cancer agent, an “therapeutically effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of Formula (I) being used by following, for example, dosages reported in the literature and recommended in the Physician's Desk Reference (57th Ed., 2003).

“Treating” or “treatment” refers to obtaining a desired pharmacological and/or physiological effect. The effect can be therapeutic, which includes achieving, partially or substantially, one or more of the following results: partially or substantially reducing the extent of the disease, condition or cancer; ameliorating or improving a clinical symptom or indicator associated with the disease, condition or cancer; delaying, inhibiting or decreasing the likelihood of the progression of the disease, condition or cancer; or decreasing the likelihood of recurrence of the disease, condition or cancer.

The following examples are intended to be illustrative and are not intended to be limiting in any way to the scope of the disclosure.

### EXEMPLIFICATION

### Example 1: Synthesis of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine [Compound (I)]

### 1.1 Synthesis of (2R,3S)-2-methyl-3-(methylsulfonylmethyl)azetidine

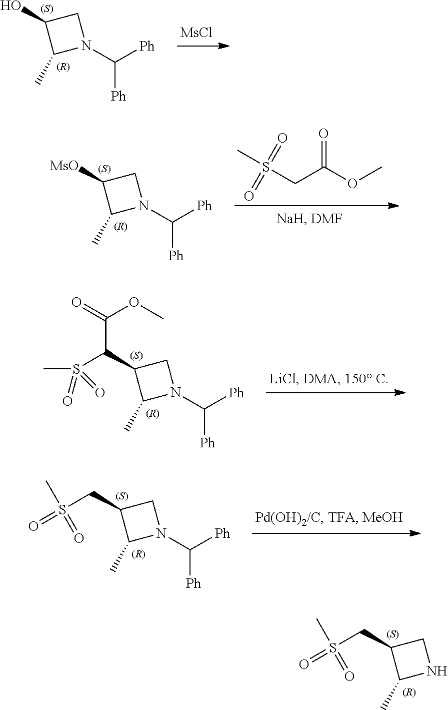

### Step 1: Synthesis of (2R,3S)-1-benzhydryl-2-methylazetidin-3-yl methanesulfonate

(2R,3S)-1-benzhydryl-2-methylazetidin-3-ol (Pharmablock, 20 g, 78.9 mmol) was dissolved in 300 mL DCM and TEA (9.55 g, 94.6 mmol) was added and the reaction mixture cooled in an ice bath. Mesyl chloride (9.93 g, 86.7 mmol) was added dropwise and allowed to stir, warming slowly to rt and stirred overnight. The mixture was diluted with DCM and washed with water and the organic phase dried over sodium sulfate, filtered and evaporated to give 26 g (98%) of the title compound as a viscous yellow oil. Analytical Data: LC-MS: (ES, m/z)=332 [M+1].

### Step 2: Synthesis of (S)-methyl 2-((2R,3S)-1-benzhydryl-2-methylazetidin-3-yl)-2-(methylsulfonyl)acetate

(2R,3S)-1-benzhydryl-2-methylazetidin-3-yl methanesulfonate (26 g, 78.4 mmol) and methyl 2-(methylsulfonyl)acetate (15.3 g, 101 mmol) were dissolved in 260 mL DMF and then NaH (3.75 g of 60% dispersion in mineral oil, 6.63 mmol) was added and stirred for ˜15 minutes, until hydrogen evolution had ceased. The reaction mixture was heated to 80° C. overnight. The reaction was cooled and then diluted with ˜200 mL water and extracted with EtOAc and combined organics washed with water, brine and dried over sodium sulfate, filtered and evaporated to give the crude product. The residue was purified by chromatography (0 to 7% MeOH/DCM). Pure fractions combined and evaporated to give 24 g (80%) of the title compound as a pale-yellow foam.

### Step 3: Synthesis of (2R,3S)-1-benzhydryl-2-methyl-3-(methylsulfonylmethyl)azetidine

(S)-methyl-2-((2R,3S)-1-benzhydryl-2-methylazetidin-3-yl)-2-(methylsulfonyl)acetate (24 g, 61.9 mmol) was dissolved in 240 mL DMA and lithium chloride (20.9 g, 495 mmol) was added and the flask put into a preheated block that was kept at 150° C. LC/MS indicated the starting material was consumed after 1.5 h. Cooled to room temperature and dilute with water, extracted with EtOAc and the combined organics washed with water, brine and dried over sodium sulfate. Filtered and evaporated to give the crude product and further purified by chromatography (0 to 5% MeOH/DCM). Pure fractions were combined and evaporated to give 19 g (93%) of the title compound as a pale-yellow foam. Analytical Data: LC-MS: (ES, m/z)=330 [M+1].

### Step 4: Synthesis of (2R,3S)-2-methyl-3-(methylsulfonylmethyl)azetidine

To a solution of (2R,3S)-1-(diphenylmethyl)-3-(methanesulfonylmethyl)-2-methylazetidine (1 9 g, 57.3 mmol) in MeOH (270 mL) was added TFA (9 mL) and Pd(OH)₂ (5.7 g), the reaction was stirred overnight at rt under H₂ atmosphere. The reaction mixture was filtered and evaporated to give the crude title compound (17 g) as a light-brown oil. Analytical Data: LC-MS: (ES, m/z)=164 [M+1].

### 1.2 Synthesis of 2-((3S,4R)-3-fluoro-4-methoxypiperidin-1-yl)pyrimidin-4-amine

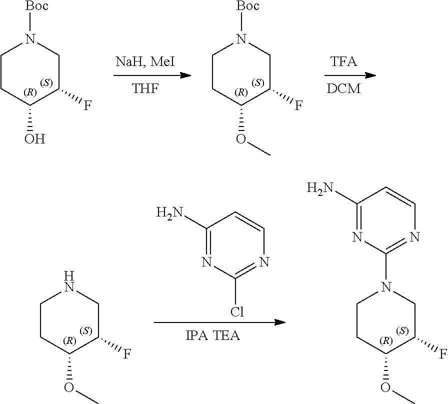

### Step 1: Synthesis of (3S,4R)-tert-butyl 3-fluoro-4-methoxypiperidine-1-carboxylate

Sodium hydride (218.90 mg, 9.122 mmol, 4 equiv.) was added to tert-butyl (3S,4R)-3-fluoro-4-hydroxypiperidine-1-carboxylate (500 mg, 2.280 mmol, 1 equiv.) in THF (10 mL) at 0° C. After stirring for 20 min, methyl iodide (1294.73 mg, 9.122 mmol, 4 equiv.) was added. The resulting solution was stirred for additional 1 h at 0° C. The reaction was then quenched by addition of 10 mL of water. The solids were filtered out. The resulting solution was extracted with EtOAc and concentrated under vacuum. This resulted in 500 mg (94.1%) of the title compound as light-yellow oil. Analytical Data: LC-MS: (ES, m/z)=178 [M+1-56].

### Step 2: Synthesis of (3S,4R)-3-fluoro-4-methoxypiperidine

The solution of tert-butyl (3S,4R)-3-fluoro-4-methoxypiperidine-1-carboxylate (500 mg, 2.143 mmol, 1 equiv.) in TFA/DCM ( 3/10 mL) was stirred for 1 h at rt. The resulting mixture was concentrated under vacuum to afford 500 mg (crude) of the title compound as a solid.

### Step 3: Synthesis of 2-((3S,4R)-3-fluoro-4-methoxypiperidin-1-yl)pyrimidin-4-amine

The mixture of (3S,4R)-3-fluoro-4-methoxypiperidine (3 g, 22.528 mmol, 1 equiv.), 2-chloropyrimidin-4-amine (2.33 g, 0.018 mmol, 0.8 equiv.) and TEA (6.84 g, 0.068 mmol, 3 equiv.) in IPA (3 mL) was stirred for 12 h at 100° C. The solvent was removed under vacuum and residue was purified by FLASH (5% MeOH in DCM) to give 3.3 g (66%) of the title compound as a light-yellow solid. Analytical Data: LC-MS: (ES, m/z)=227 [M+1]. ¹H-NMR (400 MHz, 6d-DMSO) δ ppm 7.72 (d, 1H, J=5.6 Hz), 6.39 (s, 2H), 5.71 (d, 1H, J=5.6 Hz), 4.83 (d, 1H, J=49.3 Hz), 4.60-4.49 (m, 1H), 4.29 (d, 1H, J=13.3 Hz), 3.55-3.42 (m, 1H), 3.28 (d, 1H, J=13.3 Hz), 3.20-3.04 (m, 1H), 1.76-1.48 (m, 2H)

### 1.3 Synthesis of 8-bromo-3-chloro-5-isopropylisoquinoline

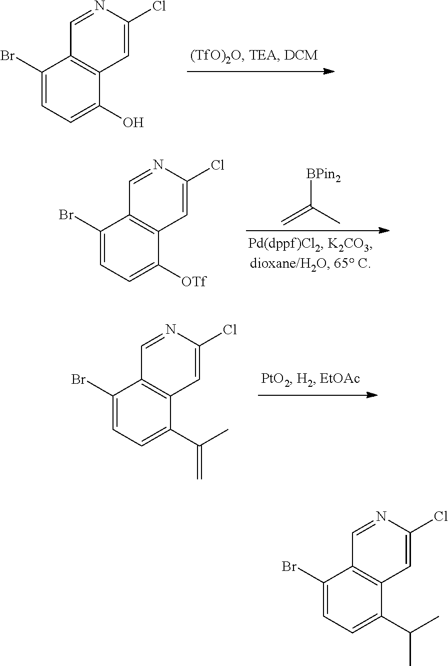

### Step 1: Synthesis of 8-bromo-3-chloroisoquinolin-5-yl trifluoromethanesulfonate

Trifluoromethanesulfonyl trifluoromethanesulfonate (45.7 g, 162 mmol) was added dropwise to 8-bromo-3-chloroisoquinolin-5-ol (14 g, 54.1 mmol) and TEA (21.8 g, 216 mmol) in DCM (400 mL) at −60° C. The resulting mixture was warmed to room temperature naturally and stirred at rt for 1 h. The mixture was concentrated under vacuum. The residue was purified by a silica gel column with PE:EA=5:1 to afford 18 g (85%) the title compound as a white solid. Analytical Data: LC-MS: (ES, m/z)=392 [M+1]; 1H NMR (400 MHz, DMSO-d6) δ 9.46 (d, 1H, J=0.8 Hz), 8.20 (d, 1H, J=8.3 Hz), 8.02 (d, 1H, J=8.4 Hz), 7.93 (d, 1H, J=0.7 Hz).

### Step 2: Synthesis of 8-bromo-3-chloro-5-(prop-1-en-2-yl)isoquinoline

The mixture of K₂CO₃ (6 g, 43.5 mmol), 8-bromo-3-chloroisoquinolin-5-yl trifluoromethanesulfonate (17 g, 43.5 mmol), 4,4,5,5-tetramethyl-2-(prop-1-en-2-yl)-1,3,2-dioxaborolane (7.30 g, 43.5 mmol) and Pd(dppf)Cl₂·CH₂Cl₂ (2.83 g, 3.48 mmol) in dioxane/H₂O (200/20 mL) was stirred for 3 h at 45° C. The mixture was diluted with 500 mL of EA and washed with brine 200 mL*2. The organic layer was dried with Na₂SO₄ and concentrated under vacuum. The residue was purified by a silica gel column with PE:EtOAc=20:1 to afford 8.0 g (67%) the title compound as an off-white solid. Analytical Data: LC-MS: (ES, m/z)=282 [M+1].

### Step 3: Synthesis of 8-bromo-3-chloro-5-isopropylisoquinoline

PtO₂ (1.7 g 7.04 mmol) and 8-bromo-3-chloro-5-(prop-1-en-2-yl)isoquinoline (7.1 g, 25.1 mmol) in EA (300 mL) were stirred under an atmosphere of H₂ balloon at rt and stirred for 1 h. The solid was filtered out. The mother solvent was concentrated under vacuum. The crude product was purified by a silica gel column with PE:EtOAc=10:1 to get 6.7 g (93%) the title compound as a brown solid. Analytical Data: LC-MS: (ES, m/z)=284 [M+1].

### 1.4 Synthesis of 3-chloro-5-isopropyl-8-((2R,3S)-2-methyl-3-((methylsulfonyl)methyl)azetidin-1-yl)isoquinoline

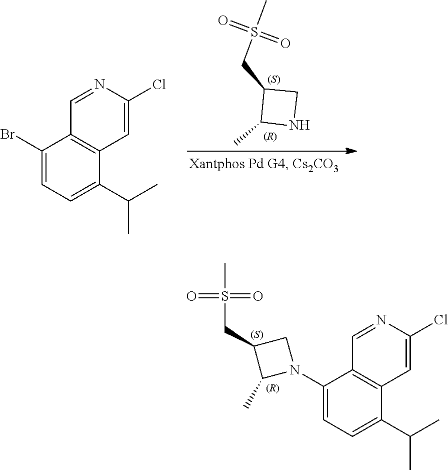

To a solution of 8-bromo-3-chloro-5-(propan-2-yl)isoquinoline (9 g, 31.6 mmol) in 1,4-dioxane (130 mL) was added (2R,3S)-3-(methanesulfonylmethyl)-2-methylazetidine (5.15 g, 31.6 mmol), Cs₂CO₃ (20.6 g, 63.2 mmol) and Xantphos Pd G4 (1.51 g, 1.58 mmol) under nitrogen. The mixture was stirred at 100° C. for 3 h under nitrogen. The reaction mixture was cooled to rt and diluted with 300 mL of water. The resulting solution was extracted with EtOAc, washed with brine, dried over anhydrous sodium sulfate and concentrated under vacuum. The crude product was purified by silica gel chromatography (0-60% EtOAc in PE) to give 7.2 g (62.6%) of 3-chloro-8-[(2R,3S)-3-(methanesulfonylmethyl)-2-methylazetidin-1-yl]-5-(propan-2-yl)isoquinoline as yellow solid. 1.5 Synthesis of N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine

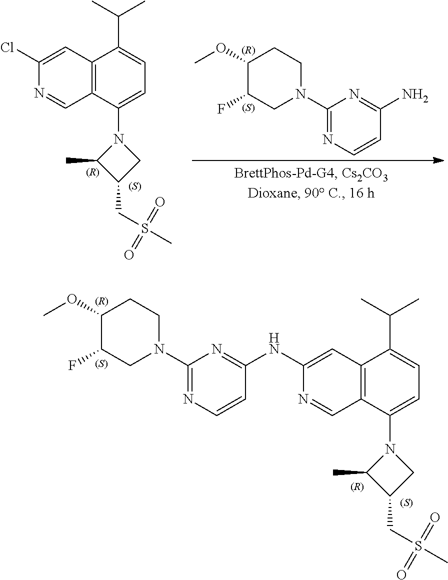

To a solution of 2-((3S,4R)-3-fluoro-4-methoxypiperidin-1-yl)pyrimidin-4-amine (18.50 mg, 0.082 mmol, 1 equiv.), 3-chloro-5-isopropyl-8-((2R,3S)-2-methyl-3-((methylsulfonyl)methyl)azetidin-1-yl)isoquinoline (30 mg, 0.082 mmol, 1 equiv.) and Cs₂CO₃ (53.3 mg, 0.164 mmol, 2 equiv.) in 1,4-Dioxane (0.82 ml) was added BrettPhos Precatalyst (Gen IV) (3.76 mg, 4.09 μmol, 0.05 equiv.) under N₂, the mixture was stirred at 90° C. for 16 h. The mixture was filtered and concentrated in vacuo. The crude mixture was purified by reverse phase chromatography (0 to 60% acetonitrile/water containing 0.1% TFA). Pure fractions were combined and neutralized with saturated sodium bicarbonate solution and then extracted with 10% MeOH/DCM (5 mL×3). Combined organic phases dried over sodium sulfate, filtered and evaporated to give 17.4 mg of the title compound (38%) as a yellow solid. XRPD diffractogram of the obtained product demonstrated that the solid was a crystalline material which was denoted as Form A.

### Example 2: Preparations of Crystalline Form B of Compound (I) Free Base

Approximately 25-35 mg of the amorphous Compound (I) was dissolved in dimethylacetamide (solvent) mostly at RT. Twice the amount of water (antisolvent) was taken in a separate vial, to which the solution was added as one transfer with rapid stirring. For example, if solids dissolved in 0.5 mL of solvent, then the solution was added to 1.0 mL of antisolvent as one transfer with vigorous stirring. Once solids were formed, the thick light-yellow slurry was filtered and washed with 1×2.0 vol. of water and left on the filter paper for 5 min with active suction from the aspirator. The sample was then placed in the oven at 50° C. under active vacuum for 15 min and then left on the benchtop overnight to dry. XRPD diffractogram of the obtained product demonstrated that the solid was a crystalline material which was denoted as Form B.

### Example 3n Compound (I) Amorphous Solid Dispersion Preparation (ASD)

Dichloromethane: Methanol (80:20) was added to a feed vessel. To this feed vessel was added Compound (I) free base (e.g., in any of the crystalline forms described herein (e.g., Forms A and B) or as an amorphous form) and hydroxypropyl methylcellulose acetate succinate (HPMCAS) in a 1:1 w/w ratio and the mixture was stirred to provide a solution and then spray dried to provide 50:50 Compound (I):HPMCAS-M SDD (spray dried dispersion).

| Component | % w/w |
|---|---|
| Compound (I) free base | 50 |
| HPMCAS | 50 |
| Solids Total | 100 |
| Methylene Chloride (Dichloromethane) | 80 |
| Methanol | 20 |
| Solution Total | 100.0 |

### Example 4. Compound (I) Formulations and 2 Capsules

| Excipients | 1 | 2 |
|---|---|---|
| 50:50 Compound (I):HPMCAS-M SDD | 50.00 | 50.00 |
| Prosolv SMCC HD90 (silicified | 20.75 | 20.75 |
| microcrystalline cellulose) | | |
| Pearlitol 50C (mannitol) | 16.25 | 15.25 |
| Kollidon CL (crospovidone) | 3.50 | 3.50 |
| Colloidal Silicon dioxide | 0.50 | 0.50 |
| Poloxamer 407 | 0.50 | 1.50 |
| Magnesium stearate | 0.50 | 0.50 |
| Intragranular | | |
| Pearlitol 50C* (mannitol) | 3.00 | 3.00 |
| Kollidon CL* (crospovidone) | 3.50 | 3.50 |
| Magnesium stearate* | 0.50 | 0.50 |
| Colloidal Silicon dioxide* | 0.50 | 0.50 |
| Poloxamer 407* | 0.50 | 0.50 |
| Extra granular total | | |
| Total | 100.00 | 100.00 |

*based on intragranular yield

The manufacturing process for Formulation 1 and Formulation 2 are as follows:
1) Intragranular components were blended in a suitably sized container using the Turbula blender for 10 minutes at 23 rpm
2) Intragranular blend was dry granulated by densification using a single station press fitted with 0.875″ tooling
3) Resultant material was milled using a Quadro Comil U5 fitted with 040G screen
4) Granule bulk density was determined and if not within 0.45±0.02 g/mL, densification and milling repeated until the desired bulk density was achieved
5) Extragranular components were blend with the granules in a Turbula blender for 5 minutes at 20 rpm
6) Final blend was hand filled into Size 4 and size 0 HPMC Capsules (Vcaps® Plus)
   Disintegration time. The time to disintegrate the capsule described as Formulation 1 and Formulation 2 was determined using USP <701> Disintegration, in particular, the procedure for uncoated or plain-coated tablets was used. Specifically, a single capsule was placed in each of the 6 tubes of the basket (Basket type A) along with the disc. Analytical grade water was added, and the temperature was maintained at 37° C.±2° C. The disintegration time for the Formulation 1 and 2 capsules was less than 15 minutes.

Dissolution Time. The dissolution time for Formulation 1 and Formulation 2 was determined using the following dissolution protocol in biorelevant media:

| Parameter | Value | |
|---|---|---|
| Apparatus | Distek 2100 C Dissolution Apparatus, USP Type II | |
| Gastric Media | 0.01N HCl | |
| (SGF) | | |
| Gastric Dose | 2000 | μgA/mL |
| Intestinal Media | 2.24 mg/mL FaSSIF in 100 mM PBS buffer, pH 6.8 | |
| (FaSSIF) | | |
| Intestinal Dose | 1000 | μgA/mL |
| Paddle RPM | 100 | |
| Bath Temp | 37.0 ± 0.5° | C. |
| Vessel Volume | 100 | mL |

| | |
|---|---|
| Centrifuged | 3 min @ 13k RPM |
| Replicates | n = 3 vessels |

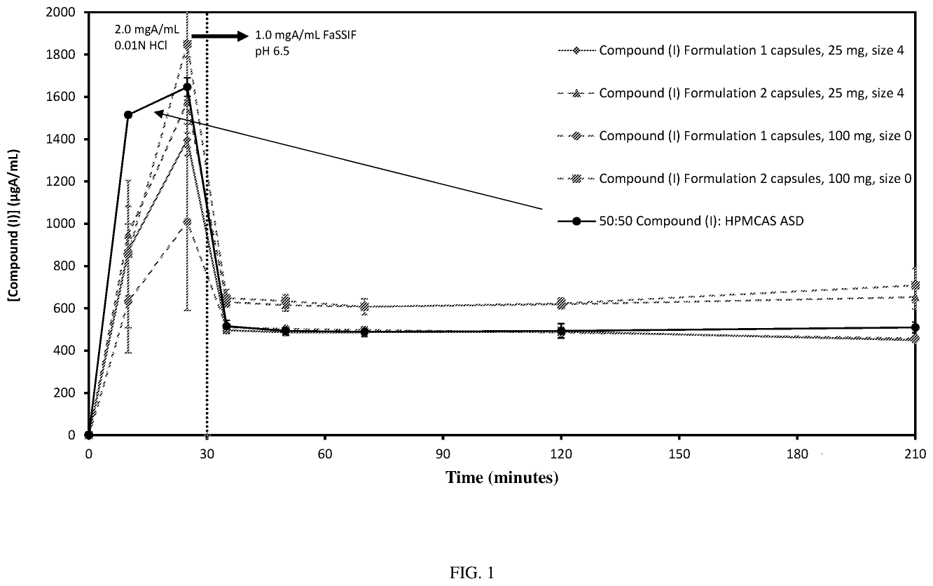

FIG. 1 is a comparison of the dissolution profile for Formulation 1 and Formulation 2 as compared to the 50:50 Compound (I):HPMCAS-M alone (i.e., the ASD alone) in biorelevant media i.e. at gastric pH and intestinal pH. Formulation 2 provided slightly higher solubility in both biorelevant medias. Formulation 1 was less soluble than Formulation 2, but still acceptable. This dissolution performance was predictive of in vivo behavior in that when these formulations were evaluated in the dog PK studies, Formulation 1 at 25 mg strength and Formulation 2 at both 25 mg and 100 mg achieved a relative bioavailability of more than 90% when compared to ASD in suspension.

### Compound (I) Tablet Formulations T1, T2, and T3

| Excipients | T1 | T2 | T3 |
|---|---|---|---|
| 50:50 Compound (I):HPMCAS-M SDD | 46.51 | 46.51 | 44.44 |
| Microcrystalline cellulose | 19.30 | 19.01 | 20.00 |
| Pearlitol 50C (mannitol) | 14.19 | 13.88 | 14.06 |
| Kollidon CL (crospovidone) | 3.26 | 3.26 | 3.50 |
| Colloidal Silicon dioxide | 0.47 | 0.47 | 0.50 |
| Poloxamer 407 | 1.40 | 2.00 | 2.00 |
| Magnesium stearate | 0.47 | 0.47 | 0.50 |
| Intragranular total | 85.6 | 85.6 | 85.0 |
| Microcrystalline cellulose* | 4.71 | 4.45 | 5.00 |
| Pearlitol 50C* (mannitol) | 4.71 | 4.45 | 4.50 |
| Kollidon CL* (crospovidone) | 3.50 | 3.50 | 3.50 |
| Magnesium stearate* | 0.50 | 0.50 | 0.50 |
| Colloidal Silicon dioxide* | 0.50 | 0.50 | 0.50 |
| Poloxamer 407* | 0.50 | 1.00 | 1.00 |
| Extragranular total | 14.4 | 14.4 | 15.0 |
| Total | 100.00 | 100.0 | 100.00 |
| Opandry II white (Nonfunctional film | N/A | N/A | 4.00 |
| coating) | | | |
| Total coated tablets | 100.0 | 100.0 | 104.0 |

*based on intragranular yield

The manufacturing process for T1, and T2 was as follows:
1) Intragranular components were blended in a suitably sized container using the Turbula blender for 10.5 minutes at 25 rpm. After addition of lubricant, components were further blended for 2.5 minutes at 25 rpm using Turbula blender.
2) Intragranular blend was dry granulated by densification using a single station press to prepare slugs (hardness: 2-5 kp and thickness about 3-4 mm)
3) Resultant slugs were gently crushed and pass through 35 mesh sieves to obtain granules.
4) Extra granular components were blend with the granules in a Turbula blender for 10 minutes at 25 rpm without lubricant. After addition of lubricant, components were further blended for 2.5 minutes at 25 rpm using Turbula blender.
5) Final blend was used to produce tablets using Korsch tablet press.

The manufacturing process for T3 was as follows:
1) Intragranular components were blended in a suitably sized blender for 250 revolutions at 15 rpm and then delumped by a comil and blend again for 250 revolutions. After addition of lubricant, components were further blended for 60 revolutions at 15 rpm.
2) Intragranular blend was dry granulated using Gerteis roller compactor at roll force 7.5 kN/cm, roll speed 1.5 rpm and roll gap 1.5 mm to form the intra granular ribbons. These ribbons were then milled using screen size at 0.8 mm to form intra granular granules.
3) Extra granular components were blended with the granules in a blender for 200 revolutions at 15 rpm without lubricant. After addition of lubricant, components were further blended for 60 revolutions at 15 rpm.
4) Final blend was used to produce tablets using Korsch tablet press.

Disintegration time. The time to disintegrate the tablet described as composition T1, T2, and T3 was determined using USP <701> Disintegration, in particular, the procedure for uncoated or plain-coated tablets was used. Specifically, a single tablet was placed in each of the 3 tubes of the basket (Basket type A) along with the disc. Analytical grade water was added, and the temperature was maintained at 37° C.±2° C. The disintegration time for the tablets was less than 1 minute 30 seconds.

Dissolution. The dissolution profiles in biorelevant media Formulations T1, T2, and T3 were determined using the method below.

Dissolution method used for T1, T2, and T3

| Solution | Preparation |
|---|---|
| Buffer | NaCl 1.999 g/L, pH 1.6 |
| FaSSGF | 2 L solution |
| pH 1.6 | Adjust the pH to 1.6 with HCl conc. |
| | Add 3.998 g of Sodium chloride |
| | Stir until totally dissolved |
| Buffer | NaCl 9.750 g/L/NaOH 1.975 g/L/ |
| Transition | NaH₂PO₄ 6.371 g/L, pH 7.5 |
| Medium | 2 L solution |
| pH 7.5 | Into a 2-L glass bottle |
| | Transfer 1800 mL of Milli-Q water |
| | Add 3.950 g of Sodium hydroxide |
| | Add 12.742 g of Sodium phosphate monobasic anhydrous |
| | Add 19.50 g of Sodium chloride |
| | Stir until totally dissolved |
| | Adjust the pH to 7.5 with HCl conc. |

Formulation T1 provided slightly longer supersaturation in FaSSIF biorelevant media. Formulation T2 cannot maintain the supersaturation longer than Formulation T1, but was still acceptable. These formulations were evaluated in the dog PK studies.

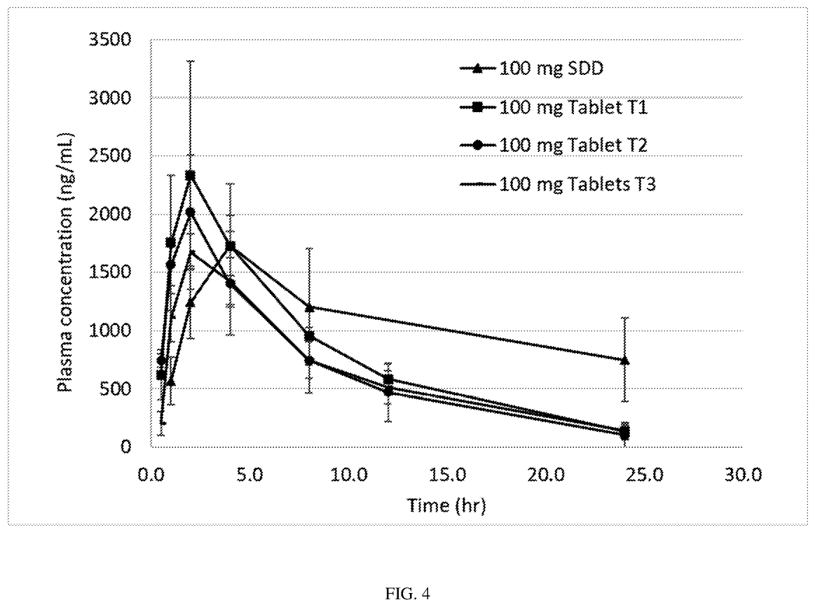

FIG. 4 is a comparison of dog PK plasma concentration profiles between 50:50 Compound (I):HPMCAS-M alone (i.e., the ASD alone), Tablet formulation T1, T2 and T3. Formulation T1 and Formulation T2 tablet at 100 mg strength achieved a relative bioavailability of more than 85% when compared to ASD in suspension. PK results of these two formulations suggested they were not significantly different from each other (p<0.05). However, higher variability was observed in Formulation T1 at the earlier timepoints as compared with T2, potentially due to the different level of total surfactant present in the formulation. PK results of Formulation T2 and T3 are similar.

### Example 5. Compound (I) Formulations 3, 4, and 5 Capsules (Comparison Formulation)

Capsule Compositions 3, 4, and 5, which include the Compound (I) amorphous solid dispersion described in Example 1, were also prepared.

| | 3 | 4 | 5 |
|---|---|---|---|
| Excipients | % w/w | | |
| 50:50 Compound (I):HPMCAS-M SDD | 50.00 | 50.00 | 50.00 |
| Prosolv SMCC HD90 (silicified | 20.75 | 20.75 | 20.75 |
| microcrystalline cellulose) | | | |
| Pearlitol 50C (mannitol) | 20.75 | 17.25 | 16.25 |
| Ac-di-sol (croscarmellose sodium) | 7.00 | 3.50 | 3.50 |
| Colloidal Silicon dioxide | 0.50 | 0.50 | 0.50 |
| Poloxamer 407 | 0.00 | 0.00 | 0.00 |
| Magnesium stearate | 1.00 | 0.50 | 0.50 |
| Intragranular total | 100.00 | 92.50 | 91.50 |
| Pearlitol 50C* (mannitol) | 0.00 | 3.00 | 3.00 |
| Ac-di-sol* (croscarmellose sodium) | 0.00 | 3.50 | 3.50 |
| Kollidon CL* | 0.00 | 0.00 | 0.00 |
| Magnesium stearate* | 0.00 | 0.50 | 0.50 |
| Colloidal Silicon dioxide* | 0.00 | 0.50 | 0.50 |
| Poloxamer 407* | 0.00 | 0.00 | 1.00 |
| Extragranular total | 0.00 | 7.50 | 8.50 |
| Total | 100.00 | 100.00 | 100.00 |

*based on intragranular yield

The manufacturing process for the Formulations 3, 4, and 5 are as follows:
1) Intragranular components (Formulations 3, 4, and 5) were blended in a suitably sized container using the Turbula blender for 10 minutes at 23 rpm
2) Intragranular blend was dry granulated by densification using a single station press fitted with 0.875″ tooling
3) Resultant material was milled using a Quadro Comil U5 fitted with 040G screen
4) Granule bulk density was determined and if not within 0.45±0.02 g/mL, densification and milling repeated until the desired bulk density was achieved
5) Extragranular components (in case of 4 and 5, not applicable to 3 as it does not contain extragranular components) were blend with the granules in a Turbula blender for 5 minutes at 20 rpm
6) Final blend was hand filled into Size 4 (25 mg strength) and size 0 (100 mg strength) HPMC Capsules (Vcaps® Plus)

Disintegration time. The time to disintegrate Formulations 3, 4, and 5 was determined as described above for Formulations 1 and 2. The disintegration time for capsules was less than 15 minutes.

Dissolution Time. The dissolution profile in biorelevant media Formulations 3, 4, and 5 was determined using the same protocol as described above for Formulations 1 and 2.

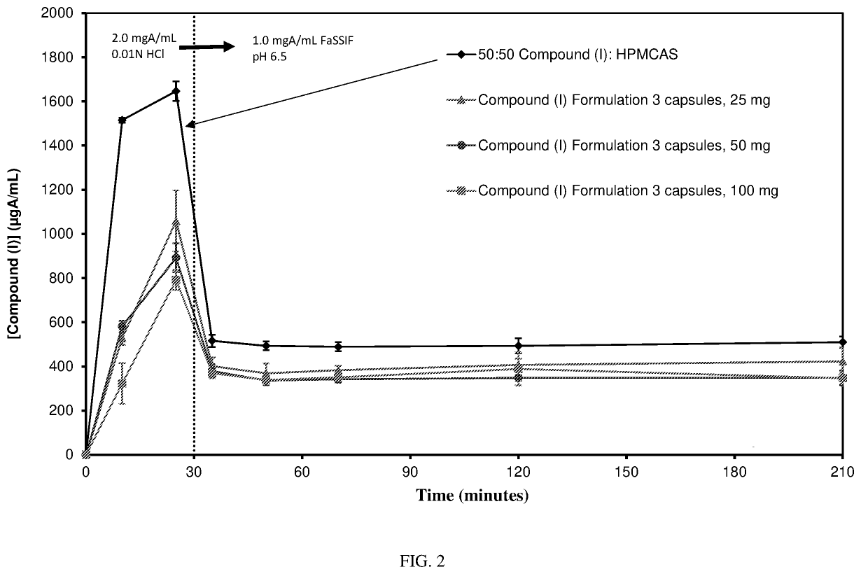

FIG. 2 is a comparison of the dissolution profile for Formulation 3 at different dosage strengths as compared to the 50:50 Compound (I):HPMCAS-M alone (i.e., the ASD alone) in biorelevant media, i.e. at gastric pH and intestinal pH. Formulation 3 performed poorly in comparison to ASD alone, as evident by the lowered solubility of Formulation 3 as compared to the ASD. Formulation 3 was also evaluated in a monkey PK studies and only 42% relative bioavailability was achieved.

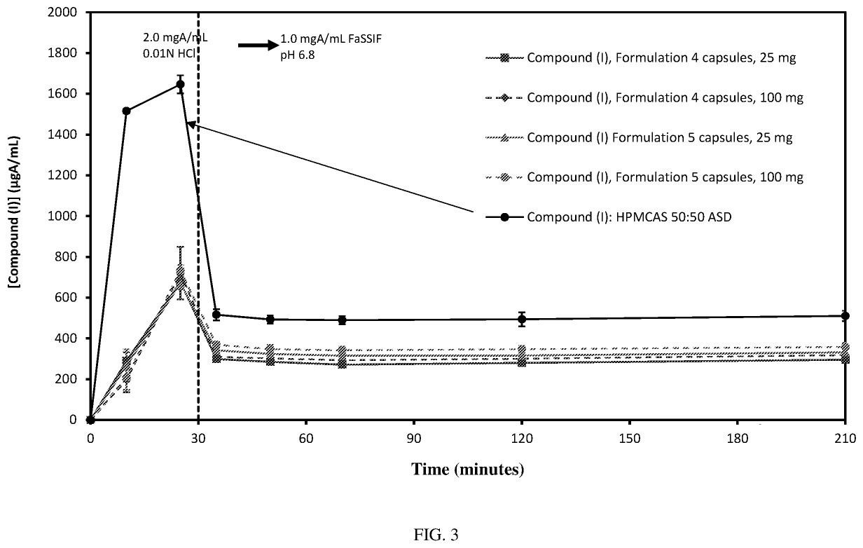

FIG. 3 is a comparison of the dissolution profile for Formulation 4 and 5 at different dosage strengths as compared to the 50:50 Compound (I):HPMCAS-M alone (i.e., the ASD alone) in biorelevant media, i.e. at gastric pH and intestinal pH. Formulation 4 and 5 performed poorly in comparison to ASD alone, as evident by the lowered solubility of Formulation 4 and 5 as compared to the ASD.

1. A pharmaceutical composition comprising:
an intragranular phase, wherein the intragranular phase comprises:
(i) an amorphous solid dispersion comprising Compound (I) or a pharmaceutically acceptable salt thereof, and a pharmaceutically acceptable polymer, and
(ii) a surfactant; and
an extragranular phase, wherein the extragranular phase comprises at least one of the following: a surfactant, a disintegrant, a glidant, a lubricant, and a filler.

2. The pharmaceutical composition of claim 1, wherein the extragranular phase comprises a surfactant.

3. The pharmaceutical composition of claim 2, wherein the surfactant in the intragranular phase and in the extragranular phase is the same.

4. The pharmaceutical composition of any one of claims 1 to 3, wherein the polymer is hydroxypropyl methylcellulose acetate succinate (HPMCAS) or polyvinylpyrrolidone/vinyl acetate copolymer (PVP-VA).

5. The pharmaceutical composition of any one of claims 1 to 4, wherein the polymer is poly(1-vinylpyrrolidone-co-vinyl acetate (or polyvinylpyrrolidone/vinyl acetate-64, PVPVA-64) or hydroxypropyl methylcellulose acetate succinate (HPMCAS).

6. The pharmaceutical composition of any one of claims 1 to 5, wherein the polymer is hydroxypropyl methylcellulose acetate succinate-M (HPMCAS-M).

7. The pharmaceutical composition of any one of claims 1 to 5, wherein the polymer is hydroxypropyl methylcellulose acetate succinate-MG (HPMCAS-MG).

8. The pharmaceutical composition of any one of claims 1 to 7, wherein the Compound (I) free base, or an equivalent amount of a pharmaceutically acceptable salt thereof, and the polymer are in a weight percent ratio of about 1:1.

9. The pharmaceutical composition of any one of claims 1 to 8, wherein the composition comprises the amorphous solid dispersion from about 20% to about 80% (e.g., about 40% to about 60%) by weight of the composition, based on the total weight of the composition.

10. The pharmaceutical composition of any one of claims 1 to 9, wherein the amorphous solid dispersion is prepared by hot melt extrusion, lyophilization, spray drying, solvent casting, or melt quenching.

11. The pharmaceutical composition of any one of claims 1 to 10, wherein the surfactant is selected from poloxamer 407, poloxamer 188 and sodium lauryl sulfate.

12. The pharmaceutical composition of any one of claims 1 to 11, wherein the surfactant is poloxamer 407.

13. The pharmaceutical composition of any one of claims 1 to 12, wherein the surfactant is poloxamer 407 micro with an average particle size of approximately 50 μm.

14. The pharmaceutical composition of any one of claims 1 to 13, wherein the surfactant is from about 0.25% to about 20% (e.g., about 0.25% to about 5% or about 0.25% to about 3%) by weight of the composition, based on the total weight of the composition.

15. The pharmaceutical composition of any one of claims 1 to 14, wherein the surfactant is in the intragranular phase from about 0.1% to about 20% (e.g., about 0.1% to about 5%) by weight of the composition, based on the total weight of the composition, and in the extragranular phase from about 0% to about 20% (e.g., about 0% to about 5%) by weight of the composition, based on the total weight of the composition, wherein the total amount of the surfactant in the combined intragranular and extragranular phases in the composition is from about 0.25% to about 20% (e.g., about 0.25% to about 10%) by weight of the composition, based on the total weight of the composition.

16. The pharmaceutical composition of any one of claims 1 to 15, wherein the surfactant is in the intragranular phase from about 0.1% to about 3% by weight of the composition, based on the total weight of the composition, and in the extragranular phase from about 0.1% to about 2% by weight of the composition, based on the total weight of the composition, wherein the total amount of the surfactant in the combined intragranular and extragranular phases in the composition is from about 0.25% to about 5% by weight of the composition, based on the total weight of the composition.

17. The pharmaceutical composition of any one of claims 1 to 16, wherein the composition further comprises a disintegrant.

18. The pharmaceutical composition of claim 17, wherein the disintegrant is selected from crospovidone, croscarmellose sodium, and sodium starch glycolate.

19. The pharmaceutical composition of claim 17, wherein the disintegrant is crospovidone.

20. The pharmaceutical composition of any one of claims 17 to 19, wherein the disintegrant is from about 0% to about 30% (e.g., about 1% to about 7% or about 5% to about 10%) by weight of the composition, based on the total weight of the composition.

21. The pharmaceutical composition of any one of claims 17 to 20, wherein the disintegrant is in the intragranular phase from about 0% to about 30% (e.g., about 0% to about 10%) by weight of the composition, based on the total weight of the composition, and in the extragranular phase from about 0% to about 30% (e.g., about 0% to about 10%) by weight of the composition, based on the total weight of the composition, wherein the total amount of disintegrant in the combined intragranular and extragranular phases in the composition is from about 0.2% to about 30% (e.g., about 0.2% to about 20%) by weight of the composition, based on the total weight of the composition.

22. The pharmaceutical composition of any one of claims 17 to 20, wherein the disintegrant is in the intragranular phase is from about 0.1% to about 5% by weight of the composition, based on the total weight of the composition, and in the extragranular phase from about 0.1% to about 5% by weight of the composition based on the total weight of the composition, wherein the total amount of disintegrant in the combined intragranular and extragranular phases in the composition is from about 5.1% to about 10% by weight of the composition, based on the total weight of the composition.

23. The pharmaceutical composition of any one of claims 1 to 22, wherein the composition comprises a glidant.

24. The pharmaceutical composition of claim 23, wherein the glidant is selected from colloidal silicon dioxide, starch, talc, tribasic calcium phosphate, powdered cellulose and magnesium trisilicate, and a mixture thereof.

25. The pharmaceutical composition of claim 23, wherein the glidant is colloidal silicon dioxide.

26. The pharmaceutical composition of any one of claims 23 to 25, wherein the glidant is from about 0% to about 5% (e.g., about 0% to about 3% or about 0.5% to about 2%) by weight of the composition, based on the total weight of the composition.

27. The composition of any one of claims 23 to 26, wherein the glidant is in the intragranular phase from about 0% to about 5% by weight of the composition, based on the total weight of the composition, and in the extragranular phase from about 0% to about 5% by weight of the composition, based on the total weight of the composition, wherein the total amount of glidant in the combined intragranular and extragranular phases in the composition is from about 0.1% to about 5% by weight of the composition, based on the total weight of the composition.

28. The composition of any one of claims 23 to 26, wherein the glidant is in the intragranular phase from about 0.1% to about 2% by weight of the composition, based on the total weight of the composition, and in the extragranular phase from about 0.1% to about 2% by weight of the composition, based on the total weight of the composition, wherein the total amount of glidant in the combined intragranular and extragranular phases in the composition is from about 0.5% to about 2% by weight of the composition, based on the total weight of the composition.

29. The pharmaceutical composition of any one of claims 1 to 28, wherein the composition further comprises a lubricant.

30. The pharmaceutical composition of claim 29, wherein the lubricant is selected from talc, calcium stearate, magnesium stearate, solid polyethylene glycols, sodium lauryl sulfate, sodium stearyl fumerate, and a mixture thereof.

31. The pharmaceutical composition of claim 29, wherein the lubricant is magnesium stearate.

32. The pharmaceutical composition of any one of claims 29 to 31, wherein the lubricant is from about 0% to about 5% (e.g., about 0% to about 3% or about 0.5% to about 2%) by weight of the composition, based on the total weight of the composition.

33. The pharmaceutical composition of any one of claims 29 to 32, wherein the lubricant is in the intragranular phase from about 0% to about 5% by weight of the composition, based on the total weight of the composition, and in the extragranular phase from about 0% to about 5% by weight of the composition, based on the total weight of the composition, wherein the total amount of lubricant in the combined intragranular and extragranular phases in the composition is from about 0.1% to about 5% by weight of the composition, based on the total weight of the composition.

34. The pharmaceutical composition of any one of claims 29 to 32, wherein the lubricant is in the intragranular phase from about 0.1% to about 2% by weight of the composition, based on the total weight of the composition, and in the extragranular phase from about 0.1% to about 2% by weight of the composition, based on the total weight of the composition, wherein the total amount of lubricant in the combined intragranular and extragranular phases in the composition is from about 0.5% to about 2% by weight of the composition, based on the total weight of the composition.

35. The pharmaceutical composition of any one of claims 1 to 34, wherein the composition further comprises one or more fillers.

36. The pharmaceutical composition of claim 35, wherein the one or more fillers are selected from anhydrous lactose or lactose monohydrate; starches including directly compressible and hydrolyzed starches; mannitol including directly compressible, spray dried and crystalline mannitols; sorbitol; xylitol; dextrose and dextrose monohydrate; sucrose-based diluents including confectioner's sugar; calcium-based diluents including monobasic calcium sulfate monohydrate, dibasic calcium phosphate dihydrate; calcium sulfate dehydrate; granular calcium lactate trihydrate; dextrans; inositol; hydrolyzed cereal solids; amylose; celluloses (including food grade sources of amorphous cellulose and powdered cellulose, microcrystalline cellulose, modified or co-processed microcrystalline cellulose, extragranular microcrystalline cellulose, and silicified microcrystalline cellulose); calcium carbonate; glycine; bentonite; polyvinylpyrrolidone; and a mixture thereof.

37. The pharmaceutical composition of claim 35, wherein the one or more fillers are mannitol and microcrystalline cellulose.

38. The pharmaceutical composition of any one of claims 35 to 37, wherein the composition comprises the one or more fillers from about 0% to about 80% (e.g., about 30% to about 50% or about 35% to about 45%) by weight of the composition, based on the total weight of the composition.

39. The pharmaceutical composition of any one of claims 35 to 38, wherein the one or more fillers are in the intragranular phase from about 0% to about 90% (e.g., about 20% to about 60%) by weight of the composition, based on the total weight of the composition, and in the extragranular phase from about 0% to about 90% (e.g., about 0% to about 20%) by weight of the composition, based on the total weight of the composition, wherein the total amount of the one or more fillers in the combined intragranular and extragranular phases in the composition are from about 5% to about 90% (e.g., about 20% to about 60%) by weight of the composition, based on the total weight of the composition.

40. The pharmaceutical composition of any one of claims 35 to 38, wherein the one or more fillers are in the intragranular phase from about 30% to about 45% by weight of the composition, based on the total weight of the composition, and in the extragranular phase from about 1% to about 10% by weight of the composition, based on the total weight of the composition, wherein the total amount of the one or more fillers in the combined intragranular and extragranular phases in the composition are from about 31% to about 50% by weight of the composition, based on the total weight of the composition.

41. The pharmaceutical composition of any one of claims 35 to 40, wherein the composition comprises two fillers, wherein the first filler is from about 15% to about 30% by weight of the composition, based on the total weight of the composition, and the second filler is from about 15% to about 25% by weight of the composition, based on the total weight of the composition.

42. The pharmaceutical composition of any one of claims 35 to 41, wherein the intragranular phase comprises two fillers selected from mannitol and microcrystalline cellulose; and the extragranular phase comprises the filler selected from mannitol.

43. The pharmaceutical composition of any one of claims 35 to 41, wherein the intragranular phase comprises two fillers selected from mannitol and microcrystalline cellulose; and the extragranular phase comprises two fillers selected from mannitol and microcrystalline cellulose.

44. The pharmaceutical composition of any one of claims 1 to 43, wherein the composition further comprises a non-functional polymer coating.

45. The pharmaceutical composition of claim 44, wherein the non-functional polymer coating is selected from hydroxypropyl methylcellulose, hydroxypropyl cellulose, hydroxyethyl cellulose, methyl cellulose, sodium carboxymethyl cellulose, polyvinyl pyrrolidone, polyvinyl pyrrolidone-polyvinyl acetate copolymer, polyvinyl alcohol, polyvinyl alcohol-polyethylene glycol copolymers, acrylic polymers, and polyethylene glycols.

46. The pharmaceutical composition of claim 44, wherein the non-functional polymer coating is selected from macrogol (PEG) PVA graft copolymer, and polyvinyl alcohol.

47. The pharmaceutical composition of claim 44, wherein the non-functional polymer coating is polyvinyl alcohol.

48. The pharmaceutical composition of any one of claims 44 to 47, wherein the composition comprises the non-functional coating polymer from about 0% to about 10% (e.g., about 0.5% to about 6% or about 2% to about 5%) by weight of the composition, based on the total weight of the composition.

49. The pharmaceutical composition of any one of claims 44-48, wherein the non-functional polymer coating includes a pigment.

50. The pharmaceutical composition of claim 49, wherein the pigment is an iron oxide-based pigment.

51. The pharmaceutical composition of any one of claims 44-48, wherein the non-functional polymer coating does not include a pigment.

52. The pharmaceutical composition of any one of claims 1 to 51, wherein the composition is prepared in an oral dosage form.

53. The pharmaceutical composition of claim 52, wherein the oral dosage form comprises (a) an amorphous solid dispersion of Compound (I) free base, or a pharmaceutically acceptable salt thereof; and a polymer; (b) a surfactant; (c) a disintegrant; (d) a glidant; (e) a lubricant; and (f) one or more fillers.

54. The pharmaceutical composition of claim 52 or 53, wherein the oral dosage form comprises (a) an amorphous solid dispersion of Compound (I) free base, or a pharmaceutically acceptable salt thereof; and hydroxypropyl methylcellulose acetate succinate (HPMCAS); (b) poloxamer 407; (c) crospovidone; (d) colloidal silicon dioxide; (e) magnesium stearate; (f) microcrystalline cellulose (MCC) and (g) mannitol.

55. The pharmaceutical composition of claim 54, wherein the intragranular phase comprises (a) an amorphous solid dispersion of Compound (I) free base, or a pharmaceutically acceptable salt thereof; and hydroxypropyl methylcellulose acetate succinate (HPMCAS); (b) poloxamer 407; (c) crospovidone; (d) colloidal silicon dioxide; (e) magnesium stearate; and (f) microcrystalline cellulose (MCC) and mannitol; and the extragranular phase comprises (b) poloxamer 407; (c) crospovidone; (d) colloidal silicon dioxide; (e) magnesium stearate; and (f) mannitol.

56. The pharmaceutical composition of claim 54, wherein the intragranular phase comprises (a) an amorphous solid dispersion of Compound (I) free base, or a pharmaceutically acceptable salt thereof; and hydroxypropyl methylcellulose acetate succinate (HPMCAS); (b) poloxamer 407; (c) crospovidone; (d) colloidal silicon dioxide; (e) magnesium stearate; and (f) microcrystalline cellulose (MCC) and (g) mannitol; and the extragranular phase comprises (b) poloxamer 407; (c) crospovidone; (d) colloidal silicon dioxide; (e) magnesium stearate; (f) microcrystalline cellulose (MCC) and (g) mannitol

57. The pharmaceutical composition of claim 52, wherein the oral dosage form comprises:
a) an amorphous solid dispersion comprising: Compound (I) free base, or an equivalent amount of a pharmaceutically acceptable salt thereof, and hydroxypropyl methylcellulose acetate succinate, wherein the Compound (I) free base or an equivalent amount of a pharmaceutically acceptable salt thereof and the hydroxypropyl methylcellulose acetate succinate are in about a 1:1 weight ratio;
b) a surfactant, and optionally,
c) a filler; a disintegrant, a glidant, and/or a lubricant.

58. The pharmaceutical composition of claim 57, wherein the oral dosage form comprises:
a) an amorphous solid dispersion comprising: Compound (I) free base, or an equivalent amount of a pharmaceutically acceptable salt thereof, and hydroxypropyl methylcellulose acetate succinate, wherein the Compound (I) free base or an equivalent amount of a pharmaceutically acceptable salt thereof and the hydroxypropyl methylcellulose acetate succinate are in about a 1:1 weight ratio;
b) a surfactant, wherein the composition comprises an intragranular phase and an extragranular phase, wherein the surfactant is present in the intragranular phase and the extragranular phase;
c) a disintegrant;
d) a glidant;
e) a lubricant; and
f) one or more fillers.

59. The pharmaceutical composition of claim 58, wherein the intragranular phase comprises (a) an amorphous solid dispersion of Compound (I) free base, or a pharmaceutically acceptable salt thereof; and hydroxypropyl methylcellulose acetate succinate (HPMCAS); (b) poloxamer 407; (c) crospovidone; (d) colloidal silicon dioxide; (e) magnesium stearate; and (f) microcrystalline cellulose (MCC) and mannitol; and the extragranular phase comprises (b) poloxamer 407; (c) crospovidone; (d) colloidal silicon dioxide; (e) magnesium stearate; and (f) mannitol.

60. The pharmaceutical composition of claim 58, wherein the intragranular phase comprises (a) an amorphous solid dispersion of Compound (I) free base, or a pharmaceutically acceptable salt thereof; and hydroxypropyl methylcellulose acetate succinate (HPMCAS); (b) poloxamer 407; (c) crospovidone; (d) colloidal silicon dioxide; (e) magnesium stearate; and (f) microcrystalline cellulose (MCC) and mannitol; and the extragranular phase comprises (b) poloxamer 407; (c) crospovidone; (d) colloidal silicon dioxide; (e) magnesium stearate; and (f) microcrystalline cellulose (MCC) and mannitol.

61. The pharmaceutical composition of claim 58, wherein the intragranular phase comprises (a) an amorphous solid dispersion of Compound (I) free base, or a pharmaceutically acceptable salt thereof; and hydroxypropyl methylcellulose acetate succinate (HPMCAS); (b) poloxamer 407; (c) crospovidone; (d) colloidal silicon dioxide; (e) magnesium stearate; and (f) microcrystalline cellulose (MCC) and mannitol; and the extragranular phase comprises (b) poloxamer 407; (c) crospovidone; (d) colloidal silicon dioxide; (e) magnesium stearate; and (f) microcrystalline cellulose (MCC) and mannitol; and a non-functional coating comprises of polyvinyl alcohol.

62. The pharmaceutical composition of any one of claims 52 to 61, wherein the oral dosage form is a capsule.

63. The pharmaceutical composition of claim 62, wherein the size of the capsule is 0.

64. The pharmaceutical composition of claim 62, wherein the size of the capsule is 0EL.

65. The pharmaceutical composition of any one of claims 52 to 61, wherein the oral dosage form is a tablet.

66. The pharmaceutical composition of any one of claims 52 to 61, wherein the oral dosage form is a tablet with a non-functional polymer coating.

67. The pharmaceutical composition of claim 65, wherein the size of the tablet is 6.1 mm round biconvex, the size of tablet is 6.35 mm round biconvex, the size of tablet is 9.0 mm round biconvex, or the size of tablet is 9.5 mm round biconvex.

68. The pharmaceutical composition of claim 65, wherein the size of the tablet is 6.9×16.9 mm oval biconvex, the size of the tablet is 8×16 mm oval biconvex, the size of the tablet is 9×18 mm oval biconvex, the size of tablet is 9.5×18.4 mm oval biconvex, the size of tablet is 10×19 mm oval biconvex.

69. The pharmaceutical composition of any one of claims 1 to 68, wherein the composition comprises about 10 mg, about 20 mg, about 30 mg, about 40 mg, about 50 mg, about 60 mg, about 70 mg, about 80 mg, about 90 mg, about 100 mg, about 125 mg, about 150 mg, about 175 mg, or about 200 mg of free base of Compound (I), or an equivalent amount of a pharmaceutically acceptable salt thereof.

70. A method for preparing the amorphous solid dispersion according to any one of claims 1 to 69, comprising: mixing the Compound (I) free base or the pharmaceutically acceptable salt thereof, with the polymer in about a 1:1 ratio; adding one or more solvents and removing the solvent(s) by heating.

71. An amorphous solid dispersion comprising Compound (I) or a pharmaceutically acceptable salt thereof, and a pharmaceutically acceptable polymer.

72. The amorphous solid dispersion of claim 71, wherein the polymer is hydroxypropyl methylcellulose acetate succinate (HPMCAS) (e.g., HPMCAS-M) or polyvinylpyrrolidone (PVP) or 6:4 linear random copolymer of N-vinylpyrrolidone and vinyl acetate (e.g., PVPVA-64).

73. The amorphous solid dispersion of claim 71 or 72, wherein the Compound (I) free base, or an equivalent amount of a pharmaceutically acceptable salt thereof, and the polymer are in a weight percent ratio of about 1:1.
